(19) United States
(12) Patent Application Publication
(10) Pub. No.: US 2024/0374433 A1
(43) Pub. Date: Nov. 14, 2024

(54) INNER EAR DEVICE WITH ACCESS AND CONDUCTIVE COMPONENTS

(71) Applicant: COCHLEAR LIMITED, Macquarie University, NSW (AU)

(72) Inventors: Daniel SMYTH, Macquarie University (AU); Wolfram Frederik DUECK, Macquarie University (AU)

(21) Appl. No.: 18/683,669

(22) PCT Filed: Aug. 20, 2022
(86) PCT No.: PCT/IB2022/057823

§ 371 Date: Feb. 14, 2024

Related U.S. Application Data
(60) Provisional application No. 63/235,312, filed on Aug. 20, 2021.

(51) Int. Cl. A61F 11/20; A61M 31/00

(52) U.S. Cl. CPC A61F 11/202; A61M 31/002; A61M 2205/04; A61M 2205/3337; A61M 2205/3507; A61M 2210/02; A61M 2210/0668

(57) ABSTRACT

A device, including a body through which a passage extends, wherein the body is configured to permanently fix to an opening in a barrier between a middle ear and an inner ear of a human, the device is an inner ear port apparatus that is configured to enable resealable physical access from the middle ear into the inner ear through the passage, and the inner ear port apparatus includes insulated electrically conductive material configured to conduct an electrical signal.

### CROSS-REFERENCE TO RELATED APPLICATIONS

This application claims priority to U.S. Provisional Application No. 63/235,312, entitled INNER EAR DEVICE WITH ACCESS AND CONDUCTIVE COMPONENTS, filed on Aug. 20, 2021, naming Daniel SMYTH of Mechelen, Belgium as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a device, comprising a body through which a passage extends, wherein the body is configured to permanently fix to an opening in a barrier between a middle ear and an inner ear of a human, the device is an inner ear port apparatus that is configured to enable resealable physical access from the middle ear into the inner ear through the passage, and the inner ear port apparatus includes insulated electrically conductive material configured to conduct an electrical signal.

In an exemplary embodiment, there is a device, comprising a tissue interface portion configured to implantably attach to tissue of and/or proximate an inner ear of a human, and a powered sensor, wherein the device is a non-simulative device configured to sense at least one phenomenon related to the inner ear of the human.

In an exemplary embodiment, there is a device, comprising a tissue interface portion configured for securement to tissue of and/or proximate an inner ear of a human and provide a long term passage from outside the inner ear to inside the inner ear and a therapeutic substance container at least indirectly releasably attached to the tissue interface portion and/or a portion of the device supported by the tissue interface portion, wherein the device is configured to actively control itself and/or to be actively controlled remotely to deliver therapeutic substance contained in the container to an inner ear, and the therapeutic substance container is configured to be located entirely within a middle ear cavity and/or the inner ear of a human.

In an exemplary embodiment, there is a system, comprising an inner ear barrier tissue interface apparatus through which a passage extends, wherein the inner ear barrier tissue interface apparatus is configured to permanently fix to an opening in a barrier between a middle ear and an inner ear of a human, and an active first component, wherein the system is configured to enable resealable physical access from the middle ear into the inner ear through the passage, the active first component is detachably attached directly or indirectly to the inner ear barrier tissue interface apparatus, and the system is configured to enable the active first component to be readily removed from the inner ear barrier tissue interface when the inner ear barrier tissue interface apparatus is permanently fixed to the barrier between the middle ear and the inner ear of the human.

In an exemplary embodiment, there is a method, comprising obtaining access, at a location within a middle ear of a human, to an implanted dedicated port configured to provide access to an inner ear from the middle ear of a human, wherein the port openably closes a passageway between the inner ear and the middle ear, wherein the port has been implanted in the human for at least one month, removing a first component that has been implanted in the human, and coupled to the port, for at least 10 days, removably attaching a second component to the port after removing the first component, and enabling the second component to execute a function in an autonomous active manner.

In an exemplary embodiment, there is an inner ear port apparatus, including an elongate tapered metallic body through which a passage extends from a proximal end of the body to a distal end of the body, wherein an outer surface of the body along a longitudinal direction of the body includes threads or ribs configured to grip bone establishing a barrier between a middle ear and an inner ear of a human to permanently fix the body to an opening in the barrier between the middle ear and the inner ear, the inner ear port apparatus that is configured to enable resealable physical access from the middle ear into the inner ear through the passage, and the inner ear port apparatus includes circuitry.

### BRIEF DESCRIPTION OF THE DRAWINGS

Embodiments are described below with reference to the attached drawings, in which:

FIG. 1 is perspective view of a human ear;

FIG. 2 is a perspective view of an exemplary cochlear stimulator implanted in accordance with an exemplary embodiment;

FIGS. 3 and 4 and 4A are schematics depicting exemplary implantable components for background purposes;

FIG. 5 is a schematic depicting an exemplary therapeutic substance delivery system for background purposes;

FIG. 6 is a schematic depicting exemplary background working ends of an embodiment that combines the embodiments of FIGS. 3 to 5.

FIGS. 7-14 and 17 are schematics depicting exemplary embodiments according to the invention;

FIG. 15 is a schematic depicting insertion of an electrode array through a port in an exemplary embodiment;

FIGS. 16 and 18 present exemplary flowcharts for exemplary methods;

FIGS. 19 to 27 depict exemplary embodiments according to the invention;

FIG. 28 is a schematic according to an automated electrode insertion device according to an exemplary embodiment; and

FIGS. 29 and 30 and 31 are schematics depicting exemplary embodiments.

### DETAILED DESCRIPTION

Merely for ease of description, the techniques presented herein are sometimes described herein with reference to an illustrative medical device, namely a cochlear stimulator, and in other instances, a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. Some embodiments include the utilization of the teachings herein to treat an inner ear of a recipient that has and/or utilizes one or more of these devices. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein can be implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, cochlear stimulator, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

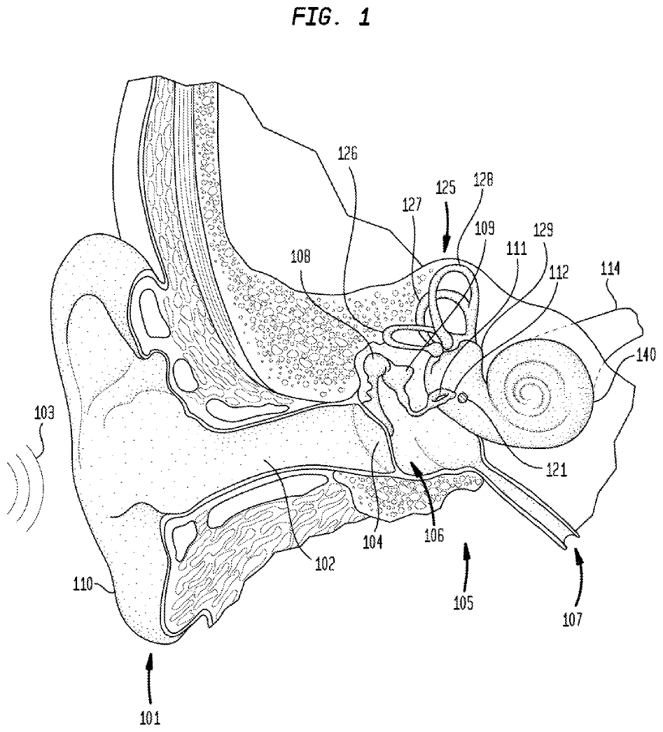

FIG. 1 is a perspective view of a human skull showing the anatomy of the human ear. As shown in FIG. 1, the human ear comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

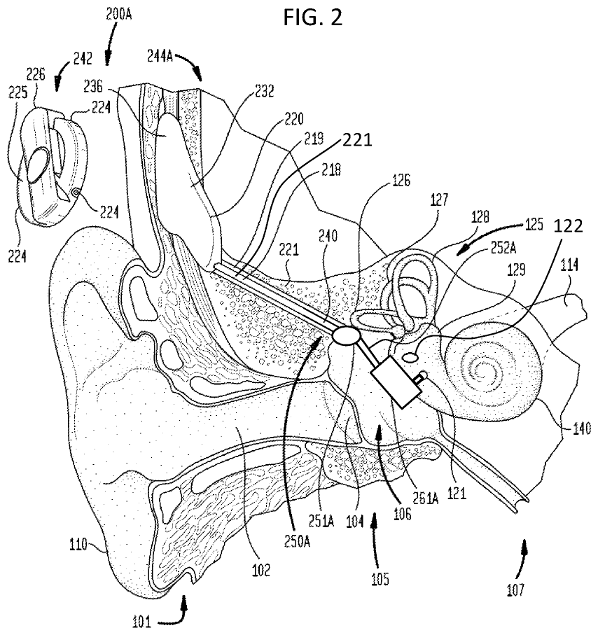

FIG. 2 is a perspective view of an exemplary cochlear stimulator 200A in accordance with some exemplary embodiments. Cochlear stimulator 200A comprises an external component 242 that is directly or indirectly attached to the body of the recipient, and an internal component 244A that is temporarily or permanently implanted in the recipient. External component 242 typically comprises two or more sound input elements, such as microphones 224 for detecting sound, a sound processing unit 226, a power source (not shown), and an external transmitter unit 225. External transmitter unit 225 comprises an external coil (not shown). Sound processing unit 226 processes the output of microphones 224 and generates encoded data signals which are provided to external transmitter unit 225. For ease of illustration, sound processing unit 226 is shown detached from the recipient.

Internal component 244A comprises an internal receiver unit 232, a stimulator unit 220, and a stimulation arrangement 250A in electrical communication with stimulator unit 220 via cable 218 extending thorough artificial passageway 219 in mastoid bone 221. Internal receiver unit 232 and stimulator unit 220 are hermetically sealed within a biocompatible housing, and are sometimes collectively referred to as a stimulator/receiver unit.

Internal receiver unit 232 comprises an internal coil (not shown), and optionally, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated platinum or gold wire. The electrical insulation of the internal coil is provided by a flexible silicone molding (not shown). In use, implantable receiver unit 232 is positioned in a recess of the temporal bone adjacent auricle 110.

In the illustrative embodiment of FIG. 2, ossicles 106 have been explanted, thus revealing oval window 122.

Stimulation arrangement 250A comprises both the distal and proximal portions of cable 218 (221 and 240), an actuator assembly 261A, an actuator mount member 251A, an actuator position arm 252A that extends from actuator mount member 251A and supports or at least holds actuator assembly 261A in place relative to the outside of the cochlea 140. In an exemplary embodiment, actuator mount member 251A is osseointegrated to mastoid bone 221, or more particularly, to the exit of artificial passageway 219 formed in mastoid bone 221.

In this embodiment, stimulation arrangement 250A is implanted and/or configured such that a portion of the actuator assembly interfaces with the round window 121, as can be seen, while it is noted that in an alternate embodiment, a portion of the actuator assembly interfaces with the oval window 122 (and both windows in some alternate embodiments).

As noted above, a sound signal is received by microphone(s) 224, processed by sound processing unit 226, and transmitted as encoded data signals to internal receiver 232. Based on these received signals, stimulator unit 220 generates drive signals which cause actuation of actuator assembly 261A.

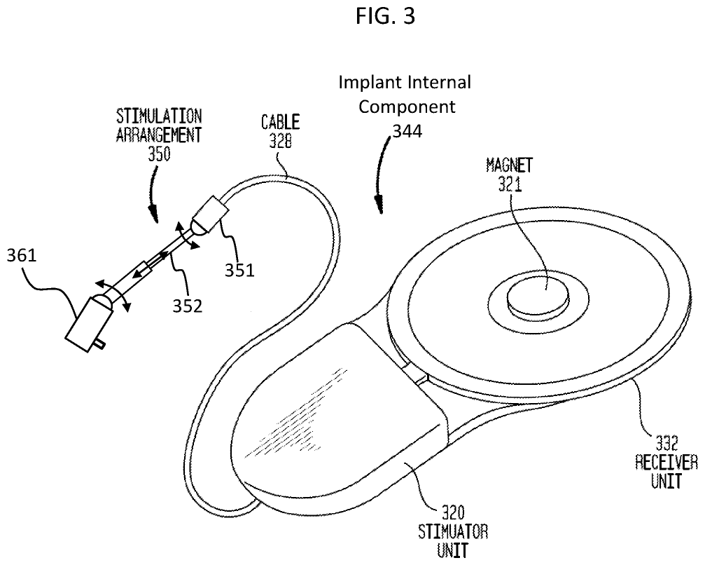

FIG. 3 is a perspective view of an exemplary internal component 344 of an implant which generally represents internal component 244A described above. Internal component 344 comprises an internal receiver unit 332, a stimulator unit 320, and a stimulation arrangement 350. As shown, receiver unit 332 comprises an internal coil (not shown), and a magnet 321 fixed relative to the internal coil. In some embodiments, internal receiver unit 332 and stimulator unit 320 are hermetically sealed within a biocompatible housing. This housing has been omitted from FIG. 3 for ease of illustration.

Stimulator unit 320 is connected to stimulation arrangement 350 via a cable 328, corresponding to cable 218 of FIG. 2. Stimulation arrangement 350 comprises an actuator assembly 361, corresponding to actuator 261A of FIG. 2, an actuator assembly mount member 351, corresponding to actuator assembly mount member 251A of FIG. 2, and an actuator assembly positioning arm 352, corresponding to the actuator assembly positioning arm 352 of FIG. 2. In an exemplary embodiment, actuator assembly mount member 351 is configured to be located in the artificial passageway 219 or adjacent thereto and fixed to the mastoid bone of the recipient. As indicated by the curved arrows of FIG. 3, the actuator assembly mount member 351 and the actuator assembly 361 are configured to enable articulation of the actuator assembly positioning arm 352 relative to those components. Further, as indicated by the straight arrow of FIG. 3, the actuation assembly positioning arm 352 is configured to telescope to provide longitudinal adjustment between the actuator assembly 361 and the actuator assembly mount member 251.

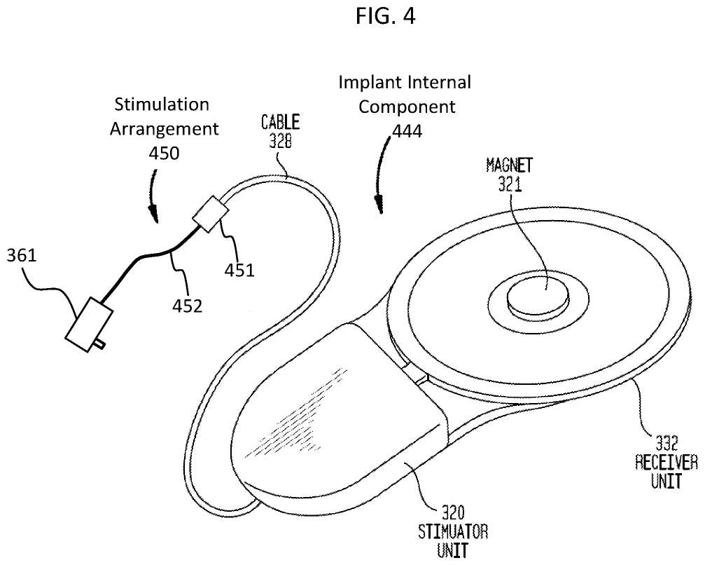

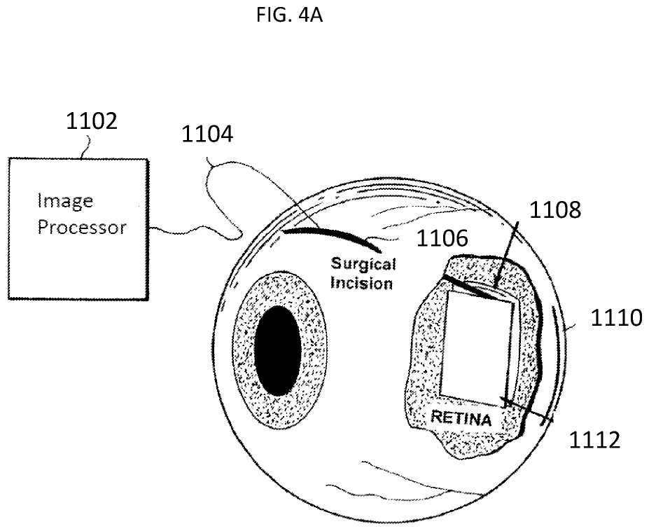

FIG. 4 is a perspective view of an exemplary internal component 444 of an implant which generally represents internal component 244A described above. Internal component 444 comprises like components corresponding to those of internal component 344.

As with internal component 344, internal component 444 is such that stimulator unit 320 is connected to stimulation arrangement 450 via a cable 328, corresponding to cable 218 of FIG. 2. However, element 451 is a coupling that instead of coupling to the articulation device detailed above in the embodiment of FIG. 3, couplies to cable 452 which is coupled to actuator assembly 361. This embodiment provides a less complicated arrangement which can have utilitarian value where the surgeon or the like is going to hand connect actuator assembly 361 directly to the exterior of the cochlea and where actuator assembly 361 will remain in place relative to the cochlea for a given period of time. The cable 452 is flexible so as to permit relative ease of movement of the actuator assembly 361 during the implantation process. The coupling 451 enables the stimulation arrangement 350 to be replaced without removing the stimulator unit 320 and/or enables the stimulator unit 320 to be removed and replaced without removing the stimulation arrangement 450.

FIG. 4A presents an exemplary embodiment of a neural prosthesis in general, and a retinal prosthesis and an environment of use thereof, in particular. In some embodiments of a retinal prosthesis, a retinal prosthesis sensor-stimulator 1108 is positioned proximate the retina 1110. In an exemplary embodiment, photons entering the eye are absorbed by a microelectronic array of the sensor-stimulator 1108 that is hybridized to a glass piece 1112 containing, for example, an embedded array of microwires. The glass can have a curved surface that conforms to the inner radius of the retina. The sensor-stimulator 108 can include a microelectronic imaging device that can be made of thin silicone containing integrated circuitry that convert the incident photons to an electronic charge.

An image processor 1102 is in signal communication with the sensor-stimulator 1108 via cable 1104 which extends through surgical incision 1106 through the eye wall (although in other embodiments, the image processor 1102 is in wireless communication with the sensor-stimulator 1108). In an exemplary embodiment, the image processor 1102 is analogous to the sound processor/signal processors of the auditory prostheses detailed herein, and in this regard, any disclosure of the latter herein corresponds to a disclosure of the former in an alternate embodiment. The image processor 1102 processes the input into the sensor-stimulator 108, and provides control signals back to the sensor-stimulator 1108 so the device can provide processed and output to the optic nerve. That said, in an alternate embodiment, the processing is executed by a component proximate to or integrated with the sensor-stimulator 1108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 1108 captures light/images, which sensor-stimulator is implanted in the recipient. In an exemplary embodiment, there is a transcutaneous communication coil that is held against a skin of a recipient via magnetic attraction to communication with an implanted component, which implanted component provides the stimulation to evoke a sight precept. In an embodiment, the teachings herein regarding magnetic attraction are utilized in such.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

Some exemplary embodiments of the teachings detailed herein enable drug delivery to the cochlea or otherwise the delivery of a utilitarian substance to the cochlea.

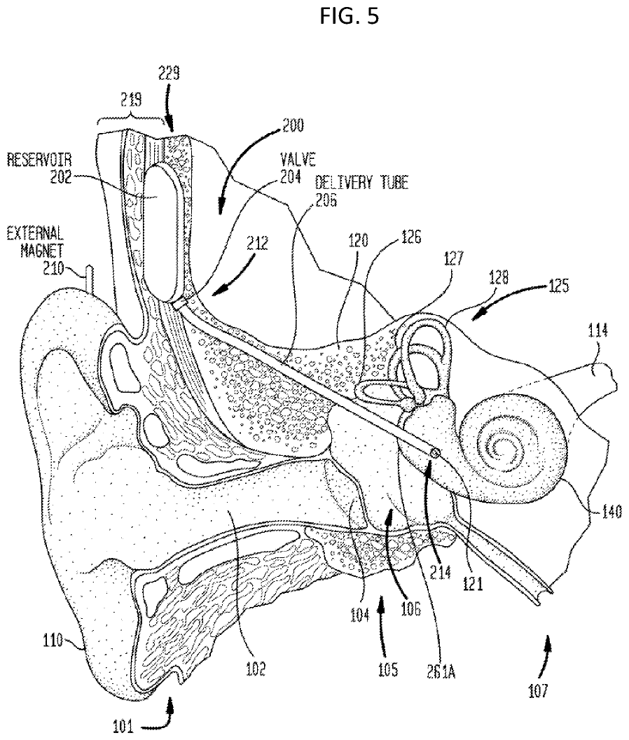

FIG. 5 depicts an exemplary drug delivery device, the details of which will be provided below. It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

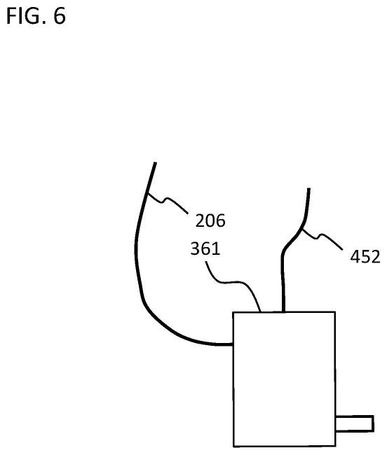

FIG. 5 illustrates an implantable delivery system 200 having an actuation mechanism, which can be modified as will be detailed below in some embodiments. However, it is noted that the delivery system 200 can also or instead have an active actuation system, again which can be modified as will be detailed below. The delivery system 200 is sometimes referred to herein as an inner ear delivery system because it is configured to deliver treatment substances to the recipient's inner ear (e.g., the target location is the interior of the recipient's cochlea 140). It is also noted that in some implementations of a modified arrangement of FIG. 5, as will be described below, the actuation mechanism enables movement of therapeutic substance to another device that in turn has an active actuation mechanism (e.g., element 361 of FIG. 6A, additional details of which are described below), where the latter is used to actually transport the therapeutic substance into a cochlea (the former is used to get the substances to the latter).

Delivery system 200 of FIG. 5 comprises a reservoir 202, a valve 204, and a delivery tube 206, in addition to some additional components, as will be described below. For ease of illustration, the delivery system 200 is shown separate from any implantable auditory prostheses. Additionally, the delivery system 200 can include, or operate with, an external magnet 210, which is separate from or part of the implantable auditory prostheses, for purposes of, e.g., controlling operation of valve 204.

The delivery tube 206 includes a proximal end 212 and a distal end 214. The proximal end 212 of the delivery tube 206 is fluidically coupled to the reservoir 202 via the valve 204.

FIG. 5, as shown, utilizes an actuation mechanism to produce a pumping action to transfer a treatment substance from the reservoir 202 to the delivery device 208 at the distal end 214 of the delivery tube 206, but again, some embodiments are modified versions of FIG. 5 that utilize active actuation.

In some implementations of FIG. 5, external force is applied on the tissue 219 adjacent to the reservoir 202 to create the external force. As will be described below, in some embodiments, an external vibratory device of a passive transcutaneous bone conduction device that vibrates to evoke a hearing percept is pressed onto the soft tissue 219 under which the reservoir 202 is located. The movement (e.g., oscillation/vibration) of the actuator causes deformations the reservoir 202 to create pumping action that propels the treatment substance out of the reservoir.

As noted, the treatment substance (sometimes herein referred to as therapeutic substance) is released from the reservoir 202 through the valve 204. The valve 204 may be a check valve (one-way valve) that allows the treatment substance to pass therethrough in one direction only.

Once the treatment substance is released through valve 204, the treatment substance flows through the delivery tube 206 to the cochlea, either directly, or indirectly via the actuator assembly 361/461. In embodiments utilizing the actuator assembly, the actuator assembly corresponds to a transfer mechanism to transfer the treatment substance from the delivery tube 206 into the cochlea 140 via the round window 121 (or oval window, or another orifice such as that established by a cochleostomy into the cochlea).

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

As noted herein, the therapeutic delivery system of FIG. 5 can be combined with a partially or fully implanted device configured to evoke a hearing percept. By way of example only and not by way of limitation, the therapeutic delivery system of FIG. 5 can be combined with the hearing prosthesis of FIG. 3 and FIG. 4. Briefly, in an exemplary embodiment, the actuator assembly 361 can be configured so as to receive or otherwise connect to the distal end of tube 206 of the therapeutic delivery system. In an exemplary embodiment of such as depicted in FIG. 6A, where the embodiment of FIG. 4 is presented by way of example, it is to be understood that the embodiment of FIG. 6 is also applicable to the embodiment of FIG. 3.

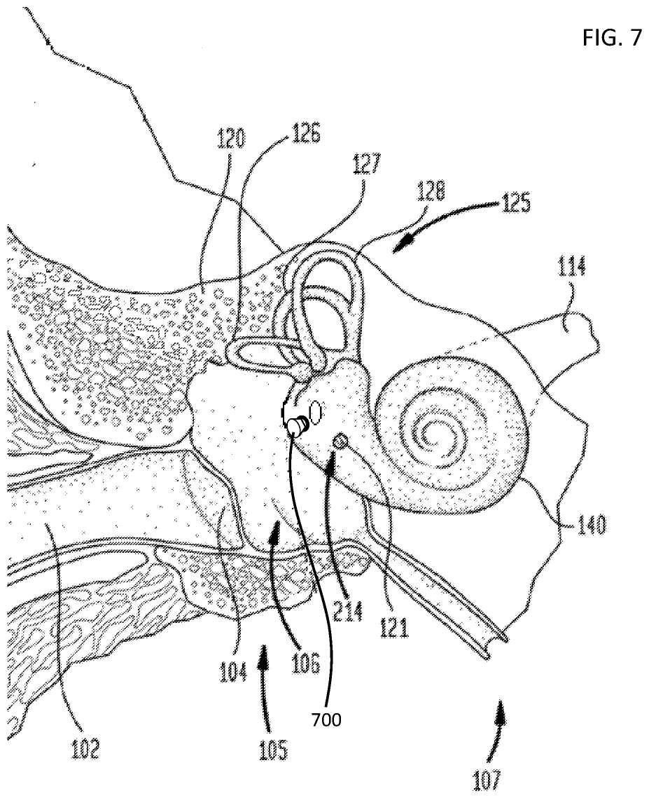

FIG. 7 presents an exemplary embodiment that is different than that disclosed in FIGS. 5-6. In this regard, the invention of this patent application corresponds to the embodiments of FIG. 7 and the figures thereafter. Any means-plus-function claims relating to the implant as a whole correspond to the structure of FIG. 7 and/or the figures thereafter. It is noted that some exemplary embodiments of the invention utilize the structure and/or function of the teachings detailed above. And embodiments of the implants according to the invention can include one or more of the above noted structures and/or functions and/or can include methods that include one or more of the above noted method actions. However, with respect to the implant, the invention does not include the implants detailed above. This is thus related art that some aspects of the invention can utilize.

It is also noted that while the teachings detailed herein are directed towards a port device or otherwise a device that establishes a passage between the middle ear and the inner ear, embodiments include utilizing the teachings detailed herein to establish a passage between other barriers of the human body between two cavities, which barriers are established by the bone for example, such as through the skull into the brain cavity, through an eye socket bone, through an arm bone or leg bone to reach the hollow portion thereof, through the rib cage to reach the heart or the lungs, etc., where a passage through the rib supports the device, etc.

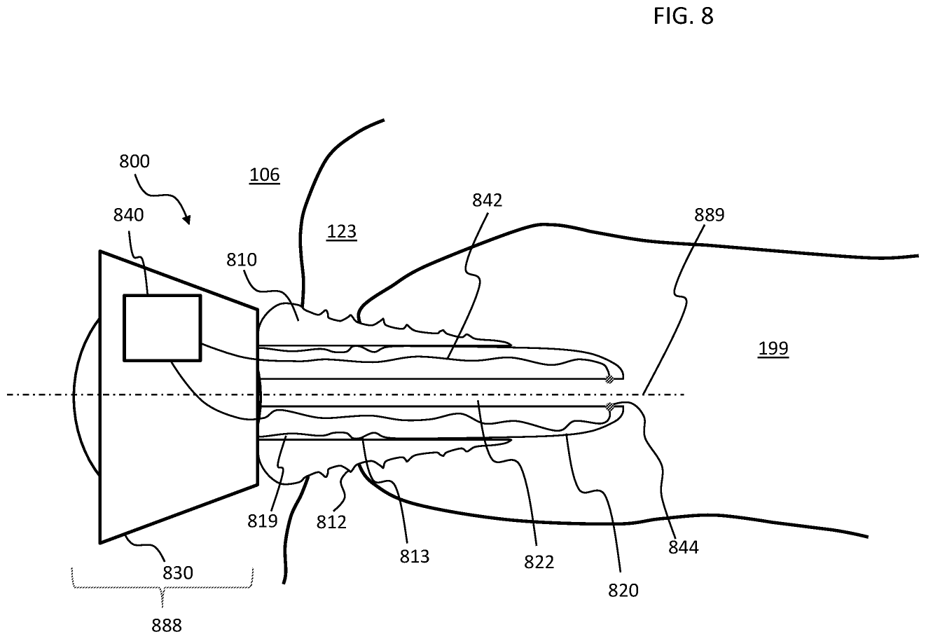

It is also briefly noted that in FIG. 7, the ossicles have been removed (from the figure) in the interest of clarity. Some embodiments can be utilized with an intact ossicles, while other embodiments are utilized in a human where the ossicles of the respective middle ear cavity have has been removed. To be clear, embodiments according to the teachings detailed herein are directed towards preserving hearing or otherwise treating hearing loss, and thus in some embodiments, the ossicles are present and functioning. But it is noted that the absence of the ossicles does not rule out embodiments associated with preserving hearing and/or treating hearing loss—hearing could be established via a middle ear implant and/or a bone conduction implant and/or a cochlear implant electrode array, etc. For example, embodiments of the teachings detailed herein can be utilized to preserve or otherwise prevent cilia degradation, where the ossicles have completely deteriorated to the point of not being useful from a medical standpoint—they might be there, but they do not function in a medically meaningful way for example. It is briefly noted by way of background that, in general, absent the teachings associated with FIG. 7 and FIG. 8 (more on this below) and variations thereof, access to the inner ear has the potential to cause damage to hearing and/or balance. Moreover, again absent the teachings associated with FIGS. 7 and 8, repeated access to try different therapies, or for repeated application of drugs, can often create added risk. For example, drug delivered to the middle ear is poorly transferred to the inner ear (thus meaning that the efficacy can be relatively low, for example). In general, providing a drug treatment to the inner ear by placing drug in the middle ear is challenging. Some access the cochlea using cochlear implant techniques and sheath introducers. Hearing drug companies often attempt to deliver to the middle ear using gels delivered to the inner ear with single shot approaches. Indeed, the standard of care for drug delivery to the ear today is middle ear injection in solution. Often, hearing drug companies try to improve delivery to the inner ear by using gels in the middle ear or by using single shot direct cochlear injection (direct in that the termination contacts directly the tissue establishing a barrier between the middle ear and the inner ear).

The teachings herein can address the above issues, at least in some embodiments. In this regard, in an exemplary embodiment, as seen in FIGS. 7 and 8, there is an inner ear port device 700. FIG. 7 depicts the visible portions of an exemplary inner ear port device 700 visible from the middle ear 106 cavity. The port device is configured to enable resealable physical access from the middle ear cavity 106 into the inner ear 199 (see FIG. 8) through a passage through the port device 700. In an exemplary embodiment, the port device 700 is configured to enable the resealable physical access at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 times or more, or any value or range of therebetween in 1 increment (e.g., 47, 66, 33 to 176, etc.). In an exemplary embodiment, the port device is configured to meet one or more of the aforementioned quantities within 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 weeks and/or months from the date of implantation of the inner ear port device into the human. Corollary to this is that there are methods of accessing the port device any one or more of the aforementioned quantities within any one or more of the aforementioned temporal periods.

At least some embodiments of the teachings herein enable inner ear access while maintaining cochlear function. At least some embodiments enable smart therapeutic substance delivery to the inner ear. At least some embodiments enable smart therapeutic substance delivery to the eye system, such as the eye shown in FIG. 4A (in an exemplary embodiment, the port devices can be implanted in an eye bone/skull bone proximate the eye and/or the nerves extending from the eye(s), and thus some embodiments can be an eye prosthesis. Some embodiments can enable implantation of such devices in the eyeball by pushing through to the vitreous humor. Accordingly, any disclosure of interfacing with an inner ear and/or providing therapeutic substance thereto and/or sensing phenomena thereof, etc., corresponds to an alternate disclosure of interfacing with body tissue associated with and/or proximate the eye(s) and/or optical nervous system, for purposes of textual economy.

Some exemplary structure of the port device 700 will now be described.

FIG. 8 depicts a side view partial cross-sectional view of an exemplary embodiment of an inner ear port device 800 which can correspond to the inner ear port device 700 noted above, which extends from the middle ear cavity 106, through the bone structure 123, that divides the middle ear cavity 106 from the interior of the cochlea 199, and thus extends therethrough. The port device can extend through the promontory. The port device can extend through the barrier between the middle ear and the inner. The port device can extend through the wall of the first turn of the cochlea. The port device can extend through the bone between the round and oval window. In this embodiment, the port device 800 includes a portion that is located in or otherwise is accessible from the middle ear cavity 106. Also as seen, the port device 800 includes a portion that is located in or otherwise is in fluid communication with the cavity 199 of the cochlea, which can be one or more of the three ducts of the cochlea. In an exemplary embodiment, therapeutic substances can be transferred from a location within the cavity 106 into the cavity 199 through the port 800.

It is briefly noted that by “transferred from a location,” this includes the scenario where the therapeutic substance travels through that area from a location that originates outside of the middle ear cavity 106. For example, a syringe including a substance can be located in the outer ear, and the termination can extend through the tympanic membrane, across cavity 106, and into the port device 800. Upon operating the syringe to transfer the therapeutic substance therein from the outer ear to the inner ear, the therapeutic substance passes through the middle ear 106, and thus is transferred from a location in the middle ear. This is as distinguished from a therapeutic substance that has as its origin location within the middle ear cavity 106, which could be the case with respect to a reservoir that is part of the port device, which reservoir is entirely located in the middle ear 106 (this would also include the species of the substance being transferred from the location within the middle ear 106—this would not include the species of the substance having an origination at the time of being attached or otherwise introduced to the body at a location outside the middle ear).

In at least some exemplary embodiments, the port device 800 is attached to the wall of the cochlea 123 at a location away from the round window and/or from the oval window. In this regard, the passage through the wall the cochlea 123 can be established via a cochleostomy through the bony structure of the cochlea 123. That said, in at least some exemplary embodiments, the port device 800 can extend through the wall of the cochlea at the location of the round window or oval window (two can be used at both locations in some embodiments), more accurately, or potentially, the former location of the round window or oval window.

FIG. 8 depicts a body 810. A passage 819 extends through that body. While in at least some exemplary embodiments, the passage 819 can include only a seal apparatus, and in this exemplary embodiment, the passage 819 has a second component, here, module 820, located therein, which module in turn has a passage 822. Body 830 is screwably attached to module 820, which body forms a head of an assembly that includes module 820 (the assembly can be considered itself a module—thus, there is a first module, body 810, and a second module that is the assembly of head 888 and element 820 (or, just element 820 can be considered the second module)). In an exemplary embodiment, pulling on the head 888 pulls out the element 820 from the passage through the body 810. In the embodiments depicted in FIG. 8, both passages extend from inside the cavity 199 to outside the cavity 106. This as compared to, for example, other embodiments where one or both of the passages extend only to the cavity 199 and/or to the cavity 106 (the passage would stop at the interface/extrapolated interface, of the cavitie(s)). This also as compared to, for example, other embodiments, where one or both of the passages do not even extend to the respective cavities, where, for example, the “end”/“beginning” of the respective passages stop short/begin after the interface/extrapolated interface, of the cavitie(s). It is also noted that the aforementioned features associated with the passages can also be applicable to the overall body configuration.

In the embodiment of FIG. 8, body 810 is configured to fix to an opening in the barrier between the middle ear in the inner ear of the human (e.g., the cochleostomy). In an exemplary embodiment, the body 810 is configured to permanently fix to an opening in the barrier.

Briefly, as seen in FIG. 8, the body 810 includes one or more protrusions 812 that can extend circumferentially about the body and/or can be located at discrete portions on the outer surface of the body 810 (e.g., they could be barbs, or spikes), and thus a combination can be utilized in some embodiments. In this embodiment, the protrusions 812 can be ribs that can have sharp edges, which will grip the bone 123 or other tissue with which the body 810 interfaces. In an exemplary embodiment, the protrusions 812 can instead be a single screw thread (and thus there would be one protrusion) and/or a plurality of screw threads, thus enabling the body 810 to be screwed into the passage. In an exemplary embodiment, the body is rotationally symmetric about the longitudinal axis 889 in its entirety (in some embodiments) and/or aside from the protrusion(s), and can be made of titanium or a titanium alloy or some other biocompatible material having sufficient longevity with respect to the intended environment (e.g., implanted as shown for 5 or 10 or 20 years, etc.). The body can be a turned and/or a casted metal body and/or 3D printed, for example, or extrusion molded, or cast molded, all by way of example. The body 810 can be turned from a thick-walled and/or a thin-walled tube. For example, a tube of titanium can be obtained, and then cut to the desired length, and then the tapered feature can be cut from the tube by turning the tube on a lathe. In an exemplary embodiment, the body 810 is a monolithic component (e.g., cut from a single tube). In an exemplary embodiment, the body can be a unified structure made from two or more components that are joined together (e.g., electrode portion of the body can be screwed into a component that establishes the right portion of the body, a “bottom” half (e.g., the portion below the axis 889) of the body can be snapped coupled to the top half of the body.

In an exemplary embodiment, the screw thread(s) of the inner ear port device can be self-tapping screw threads, more accurately, the tissue interface portion, such as body 810, of the inner ear port device can be configured as a self-tapping and/or self-threading and/or self-screwing arrangement. Any reference to self-tapping corresponds to a disclosure of an alternate embodiment of self-threading and/or self-screwing, and vis-a-versa, unless otherwise specified. Thus, embodiments include establishing a passageway through the bone between the middle ear cavity and the inner ear cavity without drilling. That is, by way of example only and not by way of limitation, at the location where the inner ear devices detailed herein and variations thereof are positioned, the first time that the barrier between the inner ear and the middle ear at that location is breached is by the inner ear port device.

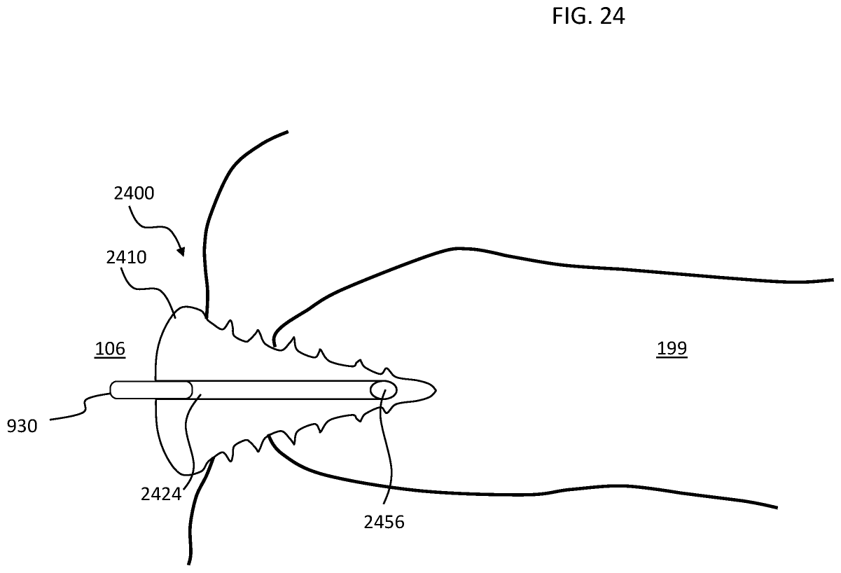

FIG. 24 presents an exemplary inner ear port device 2410 that has the self-tapping (or self-threading and/or self-screwing) features according to some embodiments. Here, there is a passageway 2424 that extends from the proximal end of the body 2410 (the body 2410 can have any one or more of the features of the body 1810 detailed above) to the outlet orifice 2456, which is located on a side of the body 2410 (the conduit established by passage 2424 and the orifice 2456 can be created by, for example, drilling only partially into the embryonic body 2410 along the longitudinal axis thereof, and then separately drilling from the side of the embryonic body 2410 at an angle of 90° to the longitudinal axes down to the end of the passage 2424) so as to provide for a “solid” the tip of the body for the purposes of self-tapping. Also shown is a plug 930, such as a temporary plug, that operates according to some of the exemplary plugs detailed herein. The plug 930 can be placed partially in the passageway 2424 as shown so as to seal the passageway, temporarily. A portion of the plug 930 can stick out of the passageway 2424 as shown, enabling the plug to be removed and/or installed by gripping the end of the plug with a tweezers and/or a forceps or tools designed for use in middle ear surgery, for example, and moving the plug accordingly.

In an alternate exemplary embodiment of a self-tapping port device, the passageway 2424 can be offset from the longitudinal axis of the body 2410. In this regard, in an exemplary embodiment, if passageway 2424 is centered at one third of the distance from the longitudinal axis and the outermost portion of the body 2410, then the opening of the passage facing the distal end would be away from the end/tip, thus enabling the self-tapping feature to be present (the passage 2424 can be completely drilled from the proximal face of the embryonic body 2410 all the way through to the other side, where, once the drill bit fully passes through the embryonic body, the tip and sufficient portions there about still remain completely intact. Thus, in some exemplary embodiments, the outlet hole can be eccentric, and, in some embodiments, the passage can be eccentric.

In an exemplary embodiment, the self-tapping feature (which can be a genus that covers self-drilling features, but can also be a species separate from self-drilling) can provide utilitarian value because there is no need to drill first or otherwise first establish a passageway through the bone separating the middle ear cavity with the inner ear cavity, because the body 2410 or any other body that has the self-tapping features establishes the passageway itself, and thus the cochlea remains fluidically sealed at all times during insertion and after the port is installed, at least until the passageway is unsealed for example. Accordingly, in an exemplary embodiment, there is the action of establishing a passageway from the middle ear to the inner ear while the cochlea remains fluidically sealed during the action of establishing the passageway and for at least 0.25, 0.5, 1, 2, 3, 4, 5, 10, 15, 20, 25, 30, 40, 50 minutes, or 1, 2, 3, 4, 5, 10, 20, 40, or 100 hours, or any value or range of is therebetween in 0.05 minute increments. In an exemplary embodiment, for at least 1, 2, 3, 4, 5, 6, 7, 8, 9, 10, 11, 12, 13, 14, or 15 months after the action of establishing the passageway utilizing the inner ear port device, the cochlea remain sealed.

Alternatively, the body could be a polymer or some form of biocompatible synthetic material. In an exemplary embodiment, the body could be made of PEEK. In an exemplary embodiment, the bodies can be casted or otherwise formed of these materials, or alternatively, cut from a larger body of these materials. In some embodiments, a coating such as Hydroxyapatite and/or titanium, can be placed over the base material of the body. Titanium coatings or some other biocompatible metallic coating can be used.

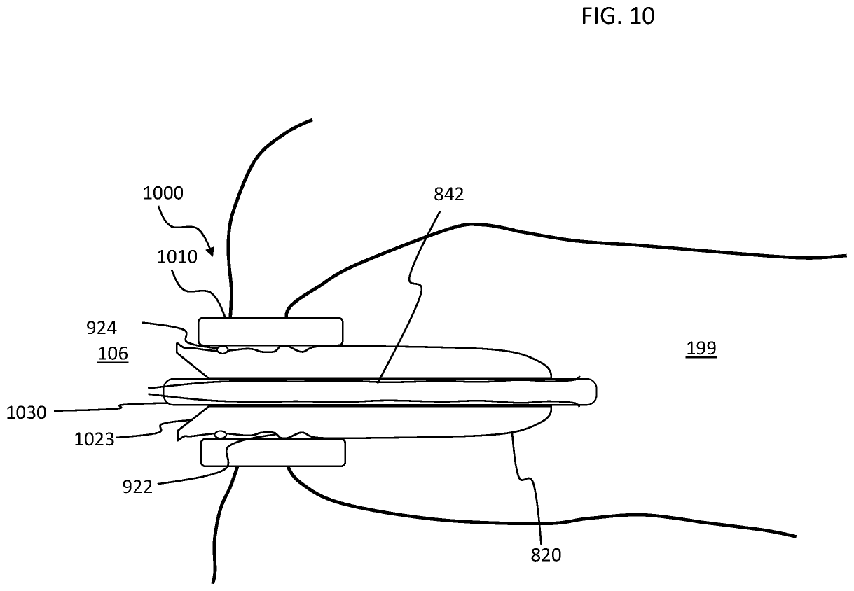

While the embodiment depicted in FIG. 8 shows a tapered body, other embodiments could have an outer surface that maintains the same distance from the longitudinal axis for at least a portion of its length, such as the portion that extends through the passage. This is seen in FIG. 10, which shows a cross-section of a body 1010 of a port 1000, which cross-section lies on it is parallel to the longitudinal axis of the body. This embodiment does not show the protrusions 812, but in other embodiments, the protrusions can be located on the outer surface. Note also and that this exemplary embodiment, an interference fit or a press fit or a strain/yield (plastic and/or elastic) fit can be utilized. In an exemplary embodiment, the outer diameter of the body 1010 can be slightly larger than the interior diameter of the passage. Utilizing a sufficiently deformable material, the body 1010 can establish the aforementioned fits and thus be secured in the passage. That said, in an alternate embodiment, the underlying deform ability of the bone can be relied upon to establish the interference fit and/or yield fit. It is noted that such fits can also be applicable to the tapered body of FIG. 8. It is also noted that such fits can be utilized in combination with the protrusions.

In an exemplary embodiment, the body could be shrunken first, such as by way of example only and not by way of limitation by chilling, and then inserted into the passage. Upon warming to body temperature, the body would then expand, and establish one of the aforementioned fits. And while the embodiments shown have the outer surface of the body that is located inside the cavity being no larger than the portions that are in the passage, in other embodiments, the portion of the body that is located inside the cavity 199 can be larger, and, in some embodiments, by utilizing the aforementioned chilling method, that larger portion could also be fit through the passage, and then upon expansion, a positive retention regime could be obtained (e.g., like a nail head). It is noted that the outer diameter of the body can also be larger than that which is in the passage with respect to portions of the body located in the cavity 106.

Any device or arrangement that can enable the functionality of the body can be utilized in some exemplary embodiments providing such is enabled by the art unless otherwise noted.

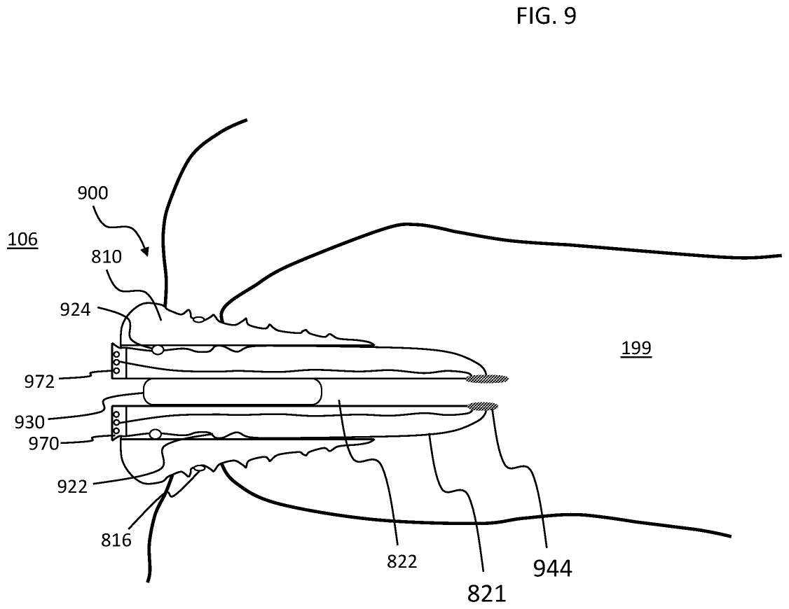

In at least some exemplary embodiments, the body 810 is configured to enable a seal between the body and the bone 123. In some exemplary embodiments, such as where, for example, there is a modicum of flexibility with respect to the structure of the body 810, the body itself can be utilized to establish the seal. In an exemplary embodiment, the protrusions 812 can be configured so as to dig into the bone and establish a seal as a result of the fact that the protrusions essentially force themselves into the bone. In an exemplary embodiment, such as where osseointegration is experienced, the osseointegration can establish a seal. Still further, as seen in FIG. 9, separate seals can be utilized, such as O-ring seal 816. Further, the sealing compound can be utilized in an exemplary embodiment, the services of the passageway that is drilled or otherwise bored through the bone 123 can be coated with a substance that will establish a seal between the body 810 and the service of the passageway through the bone 123. Any device system and/or method that can enable the establishment of a seal between the cavity 106 and the cavity 199 vis-à-vis the outer surface of the body and the bone 123 can be utilized in at least some exemplary embodiments providing that the art enables such and such presents a biocompatible way of doing so, unless otherwise noted.

And in the interest of clarity, with respect to the phrase “seal,” it is noted that that means that a barrier is established that presents a medically efficacious barrier with respect to the movement of substances from the cavity 106 to the cavity 199 and vice versa, which substances are normally expected to be found in the middle ear and/or in the inner ear, such as by way of example only and not by way of limitation, perilymph with respect to the latter, and possibly, bacteria with respect to the former (where the seal provides utilitarian value with respect to preventing bacteria that is located or otherwise present in the middle ear from reaching the inner ear all by way of example).

As noted above, some exemplary embodiments are directed towards a body that is configured to permanently fix to an opening in the barrier between the middle ear in the inner ear of a human. By “permanently fix,” it is meant that the body can remain in the human for at least a year, such as, for example, any one or more of the aforementioned longer temporal periods noted above, at the location that it is implanted at the time of implantation. This as distinguished from, for example, a temporary component/a temporary port, that might be utilized for only a few hours or a few days or a few weeks after implantation, and/or a device that could dissolve or degrade owing to the body fluids, or otherwise has a reliability engineering design such that functionality is likely to degrade to an un-functional state in a statistically significant number of designs.

And there is a middle ground, for example, where the device is configured to be “healed out” of the cochlea wall, for example. For example, the port could be designed to be pushed out by reforming bone after a few months or more (say after 3, 4, 5, 6, 7, 8, 9, 10, 11, 12 or more months). And note that this could be a scenario where the port is configured or otherwise of the design to be permanently implanted vis-à-vis the temporal periods detailed herein with respect to the mere ability to stay implanted without causing a deleterious effect on the human, but where the application thereof results in healing out of the port. But the design of the port can be configured for such result as well. That is, the port can be configured to have a shape for example that will result in the healing out of the port based on normal bone regrowth. To be clear, the two designs are not mutually exclusive to each other. The material and/or the design of the port can be configured to satisfy the longevity requirements, even though the device is not utilized for such long temporal periods.

Any reference to the features associated with longevity/permanence of the body are also applicable to one or more or all of the other components/portions of the port device unless otherwise noted, providing that the art enables such.

The fixation can be established by any of the regimes detailed above or below in an exemplary embodiment, the body is made of a material that osseointegrates to the bone, and thus in some embodiments the body is osseointegrated to the bone to achieve the aforementioned permanent fixation.

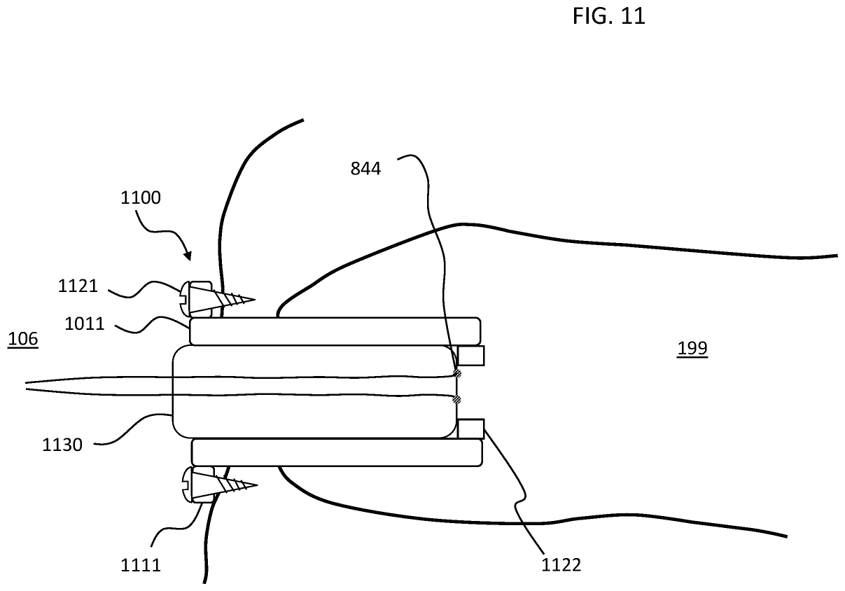

In some embodiments, bone cement or other adhesives can be utilized to permanently fix the body to the opening. In some embodiments, brackets can be utilized. For example, this can be seen in FIG. 11, where bracket 1111 is seen press fitted/interference fitted about the core of the body 1011 (collectively, element 1111 and the core 1011 make up the body-the core 1011 can be identical to the aforementioned body 1010 of FIG. 10 in some embodiments, and in others, can be different. For example, because of the bracket, a roughened surface/outer surface of the body 1010 that might be utilized to aid in the fixation might not be utilized with the core 1011 of FIG. 11, which presents an exemplary embodiment of an inner ear port device 1100 while in other embodiments, the roughened surface is utilized).

Here, the bracket has holes therethrough (not labeled) that receive one or more bone screws 1121 as seen. In an exemplary embodiment, the bone screws are what hold the body/fix the body to the passage. In an exemplary embodiment, a combination of the bone screws and an interference fit and/or a press fit with the passage through the bone 123 can be utilized. Note also that instead of bone screws and/or in addition to bone screws, bone cement or the like can be utilized, such as by way of example, by packing the bone cement between the flange 1111 and the surface of the bone 123 that faces the flange. It is noted that in at least some exemplary embodiments, the flange 1111 and the body 1011 are part of a monolithic component, which component can be turned on a lathe, from, for example, a thick-walled tube. Alternatively, the flange 1111 can be a washer type device or a ring type device which can be press fit or interference fitted on to a tube 1011.

In an exemplary embodiment, the inner ear port includes wires 842 or otherwise an electrically conductive material configured to conduct electrical current for the purposes of conducting an electrical signal. In an exemplary embodiment, the electrically conductive material can be lead wires. In an exemplary embodiment, the inner ear port includes electrodes 844 that are connected to the lead wires 842. In an exemplary embodiment, the inner ear port device can include electronics.

It is noted that in an alternate embodiment, there are no definitive separate electrodes 844 that are distinguishable from the leads. Instead, the electrodes could be the bare wires that extend into the passage 822. In this exemplary embodiment, the electrodes 844, whether they be distinct separate electrodes from leads or the ends of the leads, are electrically conductively exposed to the fluid within the cavity 199. In an exemplary embodiment, a potential between the electrodes and/or impedance between the electrodes can be measured to ascertain a latent variable that can be utilized to evaluate the perilymph within the cavity 199 for example. This could be used to determine the presence or absence of perilymph within the cavity 199 or otherwise a qualitative feature of the perilymph within the cavity 199, which can be utilized to evaluate the health of the inner ear and/or otherwise be utilized to determine whether or not a treatment regimen should be instituted, such as by way of example, providing a therapeutic substance utilizing the port device 800.

In an exemplary embodiment, the port device includes electronics package 840. This can vary from, for example, an inductance coil that can be energized from a location supercutaneous relative to the skin of the recipient (e.g., by a corresponding inductance coil located in the outer ear). In an exemplary embodiment, this can be utilized to provide current flow across the two electrodes 844 to treat the perilymph within alternating current in a treatment regimen that utilizes electrical current to change the properties of perilymph.

In an exemplary embodiment, the component supported by the body 810 is directly or indirectly releasably attached to the tissue interface portion and/or a portion of the device supported by the tissue interface portion, is devoid of active functionality.

In an exemplary embodiment, element 840 can be a computer chip and/or even a microprocessor. Element 840 can include an onboard power source that is rechargeable or not rechargeable in some other embodiments (embodiments include the utilization of batteries that can last two or three or four or five or six or seven or eight or nine or 10 years or more, which can be periodically replaced—embodiments include electronics that utilize such little power, relatively speaking, that a nonrechargeable battery can be utilized to power the system/which battery can be replaceable from the overall port or where the entire port or a portion thereof (more on this below) that includes a battery and other components can be replaced). In an exemplary embodiment, a capacitor and/or a rechargeable battery is electrically linked directly or indirectly to an inductance coil which can be utilized to transfer power to charge a recharge or otherwise create a reservoir of power that can be utilized to power the electronics/other electronics of the port.

In an exemplary embodiment, the electronics can be part of a sensor that is configured to sense one or more phenomenon, such as electrical phenomena and/or chemical phenomena and/or physical phenomena (e.g., movement of the perilymph/density of the perilymph/viscosity of perilymph, etc.).

In an exemplary embodiment, the electronics can be a memory chip. In an exemplary embodiment, the implanted port can include a connector that can enable sporadic access to the memory inside the chip at discrete points in time, where in between, the connector may not be used. In an exemplary embodiment, a medical device can be utilized to extend through the tympanic membrane or the like an opposite connector that will connect with the connector of the implanted port, where upon connection of the connectors, the data can be transferred. Some additional details of this are described below

In an exemplary embodiment, the inner ear port device according to at least some exemplary embodiments is configured to monitor one or more features related to the inner ear. Some exemplary phenomena that is monitored is described below.

In an exemplary embodiment, any disclosure herein of the electrodes 844 corresponds to an alternate disclosure where elements 844 are instead and/or in addition to electrodes, another type of sensor or any of the sensors detailed herein providing that the art enables such unless otherwise noted.

In some embodiments, simple electrodes and/or devices that measure/monitor impedance and/or monitor/measure electrical activity are utilized in at least some exemplary embodiments of the devices that utilize sensors. In some embodiments, the sensors are biosensors for detecting specific proteins or other biomolecules, or cells. In some embodiments, the sensors are configured to monitor or detect or otherwise evaluate the presence and/or absence of glucose (including determining levels), bacteria, small molecule sensors for metals, for example, or specific small molecules for drugs/artificial substances in the cochlea, and some sensors can be MIPS (molecularly imprinted polymer sensors).

Embodiments include sensors that are powered through on-board power and/or via wirelessly transmitted power. In an exemplary embodiment, the various sensors detailed herein or other types of sensors are utilized to obtain physiological data associated with a living human. In some embodiments, the implants are configured to obtain that data and evaluate that data and adjust an operation of the implant, such as, for example, changing or otherwise altering a therapeutic substance delivery regime that the implant executes when implanted. Alternatively and/or in addition to this, the data from the sensors can be transmitted to a component external to the implant, and evaluated by that component or by a human, and then control signals can be transmitted to the implant to vary the therapeutic substance delivery regime.

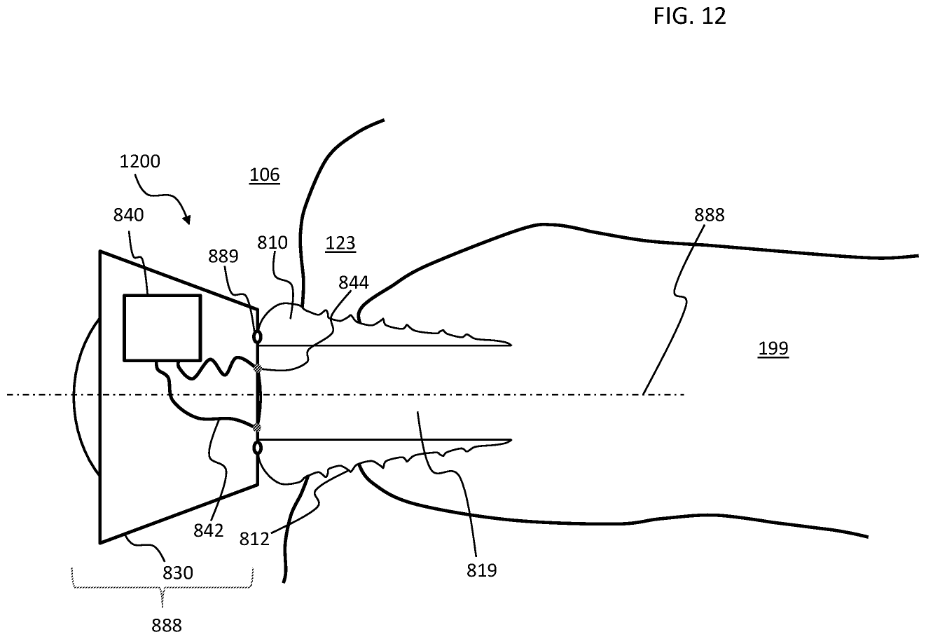

As noted above, in an exemplary embodiment, there is an inner ear device that is configured to enable resealable physical access from the middle ear into the inner ear through a passage. In the exemplary embodiment shown in FIG. 8, the passages passage 822, which is a passage through element 820, the details of which will be described below. But in some exemplary embodiments, the passage could be the passage shown that extends through the body 810 (in which element 820 is located). In an exemplary embodiment, the “head” of the inner ear port device 888 can be screwed onto and off of the body 810 by way of example. There can be seals on the head 888 that would seal the passage, and thus seal the environment of the middle ear cavity 106 from the environment of the duct 199. Accordingly, in an exemplary embodiment, there is a method of accessing the cavity 199 from the middle ear cavity 106, which includes the action of unscrewing the head 888 from the body 810 of the port device, passing a termination of a syringe through the passage 822 (which also means that the termination is passed through the passage established by the body 810 in which the element 820 is located, this can be passage 819 of FIG. 12, which is an exemplary embodiment without element 820) so that at least a portion of the termination is located within the extrapolated interior volume of the cavity 199, and then passing the therapeutic substance from the syringe through the termination into the duct 199, and then withdrawing the termination from the ports prostheses, and then reattaching the head 888 by screwing the head onto the body 810, thus sealing again the environment of the middle ear from the inner ear and vice versa. Accordingly, the head, with the utilitarian seal, enable resealable physical access from the middle ear into the inner ear through the passage. It is briefly noted that in the embodiment of FIG. 8, the action of removing the head 888 from the body 810 can be executed with the element 820 remaining in the passage 819 of the body 810, and relatively unmoved. (In an exemplary embodiment, conductive contacts can be used to place the lead portions of the head into electrical contact with the lead portions of element 820 (e.g., circular contact traces that accommodate for rotation of the head 888/misalignment when reinstalled/installed, that might exist)). Alternatively, and/or in addition to this, in an exemplary embodiment, as will be detailed below, element 820 can be structurally linked or otherwise fixed to head 888, and thus the action of removing head 888 from the body 810 can also result in the action of moving element 820 from the body 810. It is further noted that in some exemplary embodiments, such as where, for example, element 820 is a flexible body, such as, for example, a tube or sheath made of silicone, the flexible nature of the body 820 can be the feature that seals the passageway. For example, when the head 888 is screwed onto the body 810, the surface of the head facing the cavity 199 can come into contact with the opposite facing portion of elements 820, and thus compress the material of element 820, and thus establish the seal (where, for example, there is also a seal between element 820 and the inner passage of body 810).

It is again noted that while the embodiment of FIG. 7 presents a middle ear cavity 106 without ossicles, in an alternative embodiment, the ossicles are present and/or otherwise functional. This will be described in greater detail below. It is also noted that while the embodiment of FIG. 7 depicts the utilizations of the teachings detailed herein in the absence of another prosthesis and/or implant, such as a cochlear implant or a middle ear implant, it is to be noted that any disclosure herein of any embodiment associated with the port device corresponds to a disclosure of the utilization of such with any of the other prostheses detailed herein unless otherwise noted. Accordingly, in an exemplary embodiment, with respect to the embodiment of FIG. 8, for example, there is also a cochlear implant electrode array that is located in duct 199 and/or in another duct of the cochlea and/or on another side of the cochlea in the same duct (e.g., the electrode array could be located in a portion of a duct that is the portion that is proximate to, for example, the oval window, and the port device could be located at the duct that is the portion of the duct that is proximate to, for example, the round window, the electrode array could be located in a portion of a duct that is the portion that is proximate to, for example, the round window, and the port device could be located at the duct that is the portion of the duct that is proximate to, for example, the oval window).

Further, it is noted that while some embodiments of the teachings detailed herein are utilized to treat the effects associated with implanting a component in the ear system of the recipient, such as by way of example only and not by way of limitation, providing anti-inflammatory substances and/or steroids and or NSAID's and/or non-steroidal anti-inflammatory drugs to the cochlea following a cochlear implant electrode array insertion, while other embodiments of the teachings detailed herein are not utilized per se with an implant. In this regard, the teachings detailed herein can be utilized to treat hearing problems irrespective of whether or not the recipient is utilizing a hearing prosthesis. By way of example only and not by way of limitation, in an exemplary embodiment, the teachings detailed herein can be utilized to treat a syndrome that is attacking the hair cells of the cochlea prior to the utilization of a hearing prosthesis. That is, the human has not received a cochlear implant, for example, and thus is being treated to preserve the hair cells to preserve as much hearing as possible. The future recipient or otherwise the human receiving the treatment may provide the therapeutic substances himself or herself by a self-used delivery device for example. That said, the teachings detailed herein can be utilized in isolation from any other prostheses. It is also noted that the teachings detailed herein can be used in combination with conventional hearing aids. In this regard, the teachings detailed herein can be utilized to treat ailments associated with the hearing and/or balance system of a recipient that may or may not rise to the level of requiring an implantable and/or partially implantable hearing prosthesis, and the teachings herein can be utilized in combination with conventional hearing aids.

Some exemplary embodiments of the inner ear device are such that the body 810 corresponds to a first module of the implant, and the prostheses includes a second module that is removably attached to the first module, the second module configured to enable the resealable physical access. In this regard, in an exemplary embodiment, head 888 can correspond to the second module, and can be the only second module such as, where, for example, there is no element 820. Such an embodiment is schematically depicted in FIG. 12. In this exemplary embodiment, as can be seen, electrodes 844 are mounted on the head 888, and the leads 842 are truncated to be located entirely within the head 888. Because of passage 819, the perilymph or the like can reach the electrodes 844, and thus the utilitarian value of the electrodes can be harnessed even though the electrodes are technically located outside cavity 199.

In the embodiment of FIG. 12, which depicts an exemplary inner ear port device 1200, there can be an O-ring seal 889 or the like as shown, which seal provides the sealing of the passageway 819 when the head 888 is screwed onto the body 810. The O-ring seal can be physically supported by the head 888 (meaning that the O-ring moves with the head) or can be supported by the body 810. A plurality of seals can be utilized. When head 888 is screwed on to body 810, the O-ring is compressed and the seal is formed in the traditional manner.

In this exemplary embodiment, referring back to FIG. 8 for example, element 820 is structurally attached to the head 888. When the head is unscrewed from the body 810, for example, and removed from the body 810, element 820 will move in a one-to-one relationship with the head 888. In an exemplary embodiment, the head 888 can be a housing established by titanium or a polymer or some other biocompatible material, where the head can include the electronics package 840. Indeed, in an exemplary embodiment, the head 888 can be a casting of a polymer in which the electronics package and the leads (the portion of the leads that are located in the head 888) are embedded. In an exemplary embodiment, the RF inductance coil can be embedded in the polymer. It is noted that embodiments can make ample use of hermetic enclosures, such as those that can be metal and/or ceramic (e.g., ceramic/metal braze feedthroughs). Thus, any disclosure herein of any component corresponds to a disclosure of an alternate embodiment or an additional embodiment where such components are hermetically isolated or otherwise hermetically sealed from the other components and/or the ambient environment. That said, in some embodiments, hermetic sealing is not necessarily needed her otherwise always utilized. By way of example only and not by way limitation, a middle ear of a human is typically a dry space or otherwise and effectively mostly dry space, and thus non-hermetic encapsulation the electronics might be utilized.

In an exemplary embodiment, the head 888 can be made out of biocompatible silicone.

The element 820 can be a silicone body or some other body that is made of biocompatible material that can correspond to a tube with a tapered end, although in other embodiments, the end is not tapered. Any one or more of the features detailed above with respect to the body 810 can be associated with the element 820 if there is utilitarian value with respect to doing so, providing the art enables such, unless otherwise noted. In an exemplary embodiment, element 820 can establish a “plug” with respect to the interface between the outer surface of element 820 in the body 810. Briefly, in an exemplary embodiment, a protrusion or plurality of protrusions 813 can be located on the outer surface of the element 820. This can have utilitarian value in, for example, an embodiment where the element 820 is made out of a flexible or an elastomeric material. That said, the outer surface can operate in a cork like manner or the like. And in this regard, in an exemplary embodiment, by way of example, with one side of the element 820 “wet” and another side of element 820 “dry,” or any other element that would fit into the passage 819 by way of example or any the other passages herein that have the wet/dry features, swelling properties of polymers or other types of material that swell in the presence of moisture can be utilized to provide a seal or otherwise obtain a seal.

In any event, in at least some exemplary embodiments, when element 820 is located in body 810, the only way that fluid can transfer from the cavity 106 to the cavity 199 and/or vice versa is through passage 822. As noted above, element 820 can be configured as a material that has elastic deformation capabilities, which can establish a seal between the head 888 and the element 820. That said, in an exemplary embodiment, such as where, for example, element 820 (or element 821—more on this below) is a titanium tube or a titanium body or some other metallic body, or some other structure that is relatively inflexible, where, for example, O-ring seal 924 as shown in FIG. 9 is utilized to seal the passage 819 in the body 810, and O-ring analogous to O-ring 889 can be located between element 820 and the head 888 (not shown in FIG. 9—FIG. 9 depicts an embodiment of a port 900 where there is no head 888, but in alternate embodiments, there can be a head (with or without element 930, the details of which will be described below). In this exemplary embodiment, the O-ring 924 is carried by the body 821. Conversely, in an embodiment, the O-ring 824 can be carried by the body 810. While only one O-ring as shown, in an exemplary embodiment, two or more of the rings can be utilized. Other rings or other types of seals, such as a gasket for example, can be utilized.

It is briefly noted that any of the features associated with element 820 can correspond to the features associated with element 821 and vice versa unless otherwise noted providing that the art enables such.

In our continuing discussion with respect to FIG. 9, FIG. 9 depicts an exemplary second module 821, which is made of a titanium tube that has been lathed to have the taper at the end that is located in the cavity 199. Here, in this exemplary embodiment, the electrodes 944 are located at the distal most portion of the second module 821. Also as can be seen, the electrodes 944 are larger than the electrodes 844 detailed above, and are also of different sizes. In at least some exemplary embodiments, the electrodes 944 are insulated from the titanium structure that establishes the portion of the body 821 that supports the electrodes. In this exemplary embodiment, in addition to the passageway that extends from the middle ear cavity 106 to the inner ear cavity 199, the portion of module 821 that supports the electrodes 944 can be hollow. This can enable a passageway for the electrode leads extending from the electrodes 944 to the RF inductance coil 972. In this exemplary embodiment, the RF inductance coil 972 is located in a cap 970 that can be, for example, a polymer body in which the coil 972 is embedded. The cap 970 can be adhesively bonded to the titanium portion of module 821. Accordingly, the body 821 can be a composite body. The cap 970 can be made out of a material that is transparent or relatively transparent to RF inductance signals at, for example, the 5 MHz frequency.

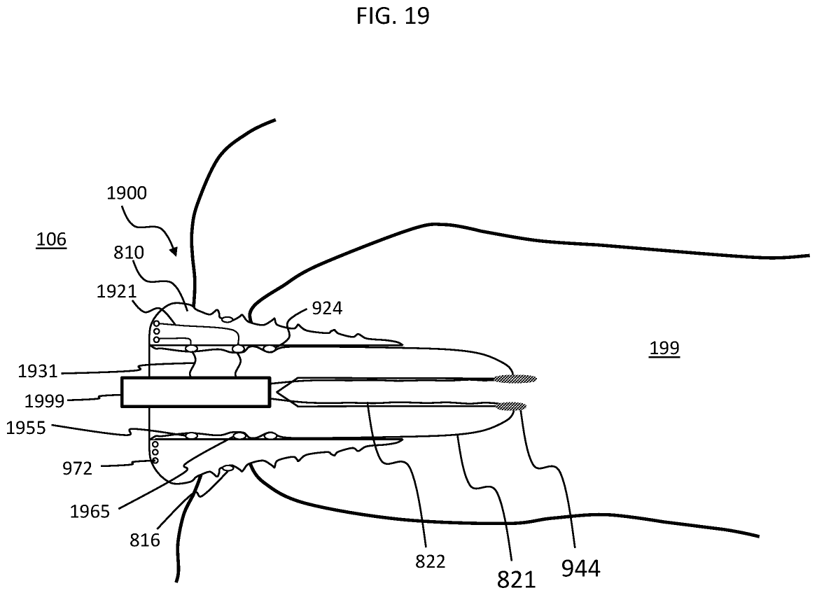

FIG. 19 presents an alternate embodiment of a device 1900 where the body 810 includes insulated conductive material (here, RF inductance coil 972, which can be made of platinum wire, and can be clad in an electrical insulative material to insulate such from the remainder of the material of the body, or, in an alternate embodiment, where the remainder of the material of the body 810 is made of an electrically insulating material, such as for example, PEEK, the inductance coil 972 can be embedded therein). Also, the leads 1921 are electrically conductive material. These leads extend within the body 810 to the O-ring's 1955 and 1965 respectively, which in this embodiment, are electrically conductive (as opposed to O-ring 924, which establishes the seal so that the perilymph cannot reach those electrically conductive O-rings), which puts the leads 1921 into electrical conductivity with the leads 1931, which are in electrically conductivity with assembly 1999, which includes a control chip and a memory chip and a power source (a long use battery), where assembly 1999 is in electrical conductivity with the leads 822 (here, the passageway in body 821 is bored only partially into body 821 so there is no need to seal the passageway, aside from sealing around the leads where the leads extend to the assembly 1999). Here, the assembly 1999 can receive inputs from the electrodes, and can output signals that are provided to the inductance coil 972, so that the inductance coil creates an inductance signal that can be read from another device, which inductance signal contains data relating to the sense phenomenon. And note that the arrangement of FIG. 19 can also be representative of a simulative device. Here, the inductance coil 972 can receive an inductance signal from another device, and a signal is provided to the assembly 1999, which can instruct the assembly on how to provide stimulation with the electrodes or otherwise enable assembly 1999 to provide stimulation via the electrodes. With respect to the former, the data received from the coils 972 can be stored in the memory device of the assembly 1999, which can be a chip memory, or a latch memory or a register memory, and the assembly 1999 can refer to that memory to determine how to operate/stimulate. Alternatively, and/or in addition to this, the data received by coil 972 can be data that enables the assembly 1999 to operate in the first instance (e.g., it can be an instruction to commence operation), where the onboard chips of the assembly 1999 enable the implant to operate autonomously (once enabled).

Embodiments can include other types of electrical contacts beyond the O-rings 1965 and 1955. In an exemplary embodiment, pin and receptacle sockets can be utilized. Any type of electrical contacts that can enable electrical power and/or data transfer between the permanent port device and the removable/replaceable component can be utilized in at least some exemplary embodiments.

It is noted that the assembly that makes up part of the inductance coil can also include a capacitor so that the coil is a tuned coil.

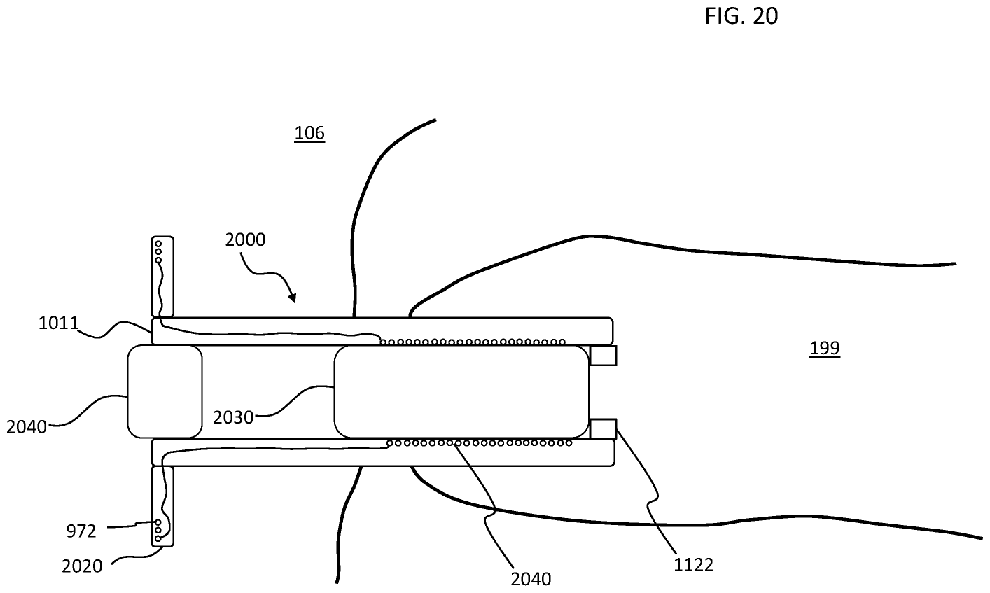

FIG. 20 presents another exemplary embodiment of an inner ear port implant 2000. Here, the electrically conductive material that is insulated is entirely located on/in the body that interfaces with the tissue (analogous to body 820 above). Here, the body is established by tube 1011 and flange 2020. The flange 2020 supports/contains the inductance coil 972, which is connected via leads to coil 2040 (a capacitor may be located between), along with other circuitry, which can enable the teachings herein, more on this in a moment). In this exemplary embodiment, the coil 2040 can be configured to generate a magnetic field which influences a therapeutic substance within container 2030, which makes the therapeutic substance contained therein more likely to elute through the container 2030. (Plug 2040 is provided to “cap” the tube 1011—in some embodiments where the container 2030 is configured so that the therapeutic substance can only elute through one end (the end facing the interior of the cochlea), the plug 2040 may not be present). In an exemplary embodiment, the reservoir 2030 can be filled with a therapeutic substance containing magnetic particles. The external magnetic field moves the particles from inside the reservoir across a membrane and/or a valve and/or an exit port, into the duct of the cochlea. The strength and/or duration of the external magnetic field controls how much therapeutic substance is administered and/or raped thereof. This can be of utilitarian value with respect to repeated therapeutic substance administrations. In an alternate embodiment, the coil 2040 is configured to generate heat, which heat is conductively transferred to the container 2030, which heating causes a propensity of the therapeutic substance therein to elute out of the container 2030. Insulation can be provided (thermal insulation) outboard of the coil 2040 for heat transfer management.

The embodiment of FIG. 20 can have utilitarian value with respect to placing the coil 972 at a location closer to the tympanic membrane/further away from the wall establishing the cochlea. This can enhance the ability of the coil 972 to receive and inductance signal originating from the outer ear for example (because it is closer thereto) relative to some of the embodiments above, where the coil is closer to the inner ear. In an exemplary embodiment, such as to provide stability, a second flange can be utilized, such as that associated with the embodiment of device 1100 disclosed above. This can be utilized to counteract any cantilever beam/leverage phenomenon that results from having the tube extend so far away from the cochlea wall.

In view of the above, it can be seen that in at least some exemplary embodiments, the insulated electrically conductive material configured to conduct an electric signal is located on or in the body that interfaces with the tissue of the cochlea. It can further be seen that in an exemplary embodiment, the electronics of the implant are located on or in the body that interfaces with the tissue of the cochlea. In an exemplary embodiment where the implant is a two module device (the body/part that interfaces with the tissue vs. the module that is removable or replaceable while the body is implanted/remains in contact with the tissue), all of the electrically conductive material that is insulated that is configured to conduct an electric signal and/or the electronics are located on the part that interfaces with the tissue and/or no such components are located on the second module/removable module.

And while the embodiment of FIG. 20 is presented in terms of a therapeutic substance delivery device, in other embodiments, the electronics can be a sensor system and/or an actuator system and/or a stimulator system by way of example only and not by way of limitation. In an exemplary embodiment, the passageway through tube 1011 can instead be sealed with a component that is of a configuration that enables access through the tube into the cochlea. By way of example only and not by way of limitation, instead of container 2030 with respect to FIG. 20, there may only be plug 2040 within the tube, and plug 2040 can be removed if access into the cochlea is desired (such as to inject the therapeutic substance into the cochlea utilizing a syringe).

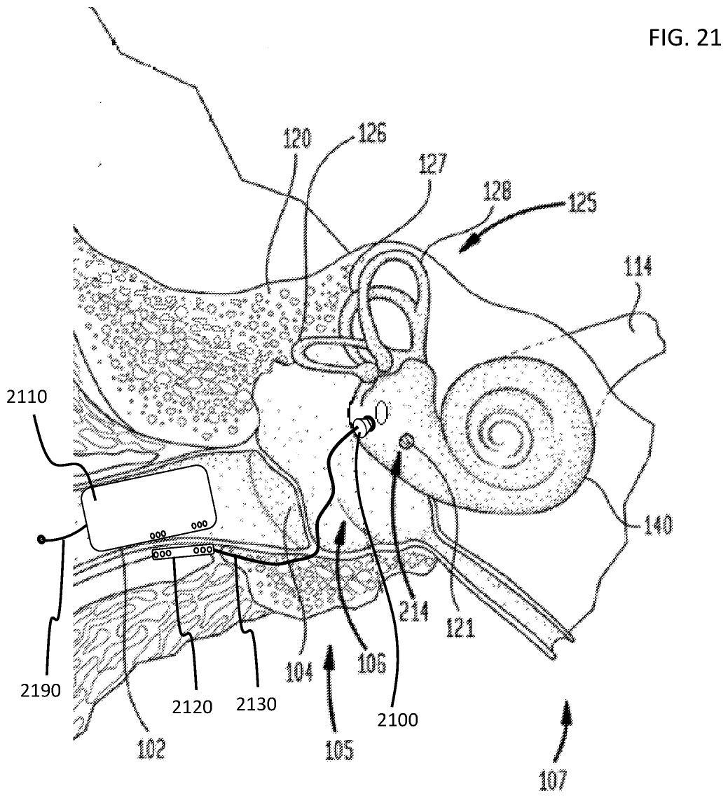

FIG. 21 presents an exemplary embodiment that includes a system that includes an in-the-ear (ITE) device 2110. The ITE device 2110 can include a transmitter, receiver, and/or a transceiver. The ITE device is depicted as being located in the ear canal 102 (where the ossicles have been removed for clarity—the ossicles may or may not be present depending on some embodiments). Here, there is an active component 2100 of the system, that can correspond to the port device that includes a module that has an active component, such as a cochlear implant electrode array assembly, or a drug delivery device, or an active sensor. The active first component or a device implanted in the human and in signal communication with the active first component (for example, where the port body 810 includes a receiver and/or transmitter and/or transceiver in total or at least one or more portions of those components) includes a receiver, transmitter and/or a transceiver apparatus and the associated antenna componentry. In an exemplary embodiment, such as this exemplary embodiment, the active component includes a wireless receiver. The active component in the ITE device are in signal communication via an inductance link established by coils of the ITE device 2110 that are configured to communicate with the implanted coils of an implanted assembly 2120 that is implanted beneath the surface of the ear canal as shown. The assembly includes an electrical lead 2130 that extends from the assembly 2120 located proximate the ear canal along the ear canal underneath the skin, and then extending across the middle ear cavity 106 to the active component 2100 of the port device. The system depicted in FIG. 21 is such that the ITE device is configured to control operation of the active component via the transcutaneous induction communication link between the transmitter and the receiver. In this regard, the active first component need not be a component that is configured to autonomously operate, although in other embodiments, this can also be the case. And note that this arrangement can also be utilized to instead enable the autonomous operation of the active component.

In an alternate exemplary embodiment, the ITE device is configured to transmit data to the implant 2100 utilizing the aforementioned wireless link. In an exemplary embodiment, this could entail providing an update to the instructions that are utilized to implement autonomous operation. In an exemplary embodiment, the active first component or otherwise the implant is configured to transmit the data signal from the active component to the ITE device. This can be data indicative of the various measurements taken by the onboard sensors, or this could be data indicative of a level of a therapeutic substance remaining, usage rates of the therapeutic substance, etc. In an exemplary embodiment, the ITE device can be utilized periodically to provide data and/or programming instructions to the implant and vice versa. That is, the ITE device can be a device that is utilized for less than all the time that the implant is operating. In an exemplary embodiment, the ITE device is located in the ear less than 20, 15, 10, 9, 8, 7, 6, 5, 4, 3, 2, or 1% of the time that the active component is functioning or any value or range of values therebetween in 0.1% increments. It is also noted that in an alternative embodiment, instead of a true ITE device as shown, the portion located in the ear canal can be a temporary communicative device, such as a wand that can be inserted into the ear canal that has an antenna at the end that can enable the communication when present, for the limited times needed.

In an exemplary embodiment, the aforementioned temporary device could be utilized in a clinical setting and/or in a home setting or otherwise in a setting away from the healthcare provider with the clinic. In this regard, a wand or other device can be issued or otherwise provided to the recipient and the recipient can be instructed on how to use such, or otherwise a caretaker or loved one or a provider of aid to the person can be trained in how to use such. Embodiments thus include methods of doing so. Further, it is noted that the ITE component can be utilized for normal use and/or use away from the clinic, but when the person goes to the doctor or other healthcare provider, the doctor or healthcare provider can utilize the wand/temporary device.

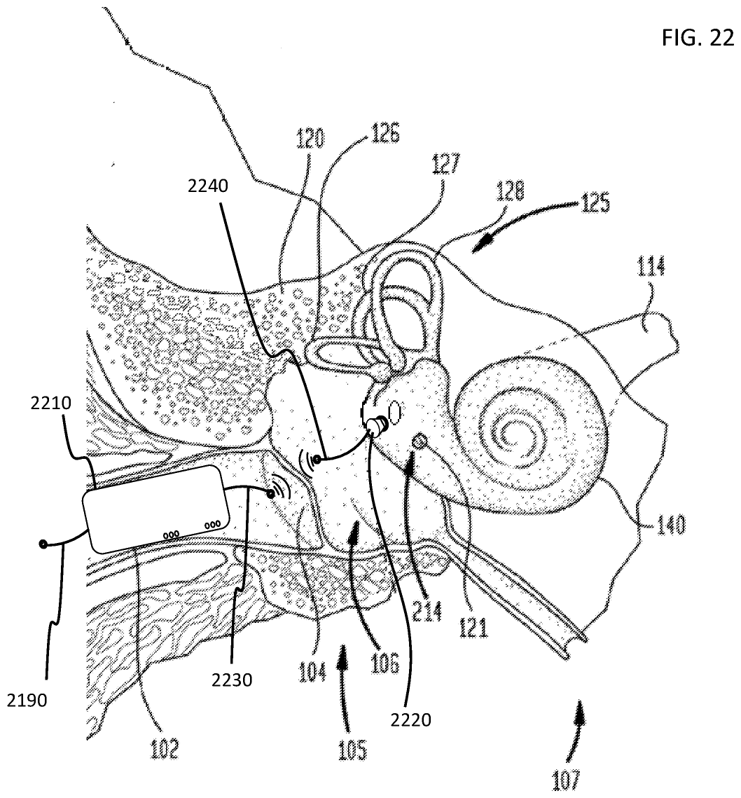

FIG. 22 presents an alternate exemplary embodiment of communication with the active components of the system. Here, the implanted device 2220 includes a more conventional antenna 2240 (not an inductance coil antenna in this embodiment). This communicates with an antenna 2230 that is part of ITE device 2210. This communication can be Bluetooth or can be a higher frequency RF communication regime (higher than that of the inductance coil embodiments, which embodiments can be about 5 MHz for example). Still, it is noted that the ITE device 2210 can include the inductance coil antenna as shown. This can enable the ability to communicate with both of the communication regimes seen in FIG. 21 and FIG. 22.

It is briefly noted that the embodiments detailed above with respect to the inductance coil can be utilized to transfer power and/or data wirelessly. This can be utilized to recharge an implanted/implantable power storage device. Of course, as just noted, the link can be utilized to directly enable smart features and/or to directly control or operate active features/active components of the implanted portion.

And while the embodiments above have focused on wireless RF communication, in an alternate embodiment, wireless optical power and/or data transfer can be implemented. A light receiving diode can be positioned with the port device/be part of the port device to enable coupling with a light-emitting diode placed in the ear canal. In an exemplary embodiment, the tympanic membrane can be sufficiently transparent to the light emitted by the light-emitting diode. Accordingly, any disclosure herein of RF communication corresponds to a disclosure of an alternate embodiment utilizing like communication.

But in any event, again returning to the concept where the inner ear port includes a first and second module, it can be seen that in at least some exemplary embodiments, removal of the second module from the first module can establish the action of unsealing the established seal, to enable the physical access from the middle ear into the inner ear through the passage, whether that passage be the passage 819 and/or passage 822. And it is further noted that in some exemplary embodiments, such as where for example, the head 888 can be removed from the body 810 and the body 820, where the body 820 could also be removed from the body 810 (but may not be done so in some exemplary methods), and thus body 820 could be a third module by way of example, albeit this third module might not be one that is configured to enable the resealable physical access per se. Still, there can be utilitarian value with respect to enabling the removal of such to enable the ultimate replacement of body 820 to upgrade the port device and/or to otherwise address a wear scenario. With respect to the former, in an exemplary embodiment, an improved sensor could be swapped out by replacing the body 820. In an exemplary embodiment, the electrodes 844 could be subject to dissolution over time owing to the exposure to the perilymph, which could be incurred by the application of electrical charge. Alternatively, and/or in addition to this, situations can change where, instead of, sensing one phenomenon or otherwise measuring one parameter, there is utilitarian value with sensing another different phenomenon were otherwise measuring another different parameter, and thus the sensors can be swapped out.

And in this regard, it is noted that in at least some exemplary embodiments, such as where the head 888 is removable from the body 820, there can be contacts to permit electrical connection between the leads 842 that are supported by the body 820 and the leads 842 that are supported by the head 888. A male-female coupling relationship to be used, such as, in some exemplary embodiments where the head 888 is snapped coupled to the body 820 and/or to the body 810. A racetrack arrangement made of conductive material can be utilized where the head 888 can be screwed onto the body 810 for example, but it is the fact that there is a racetrack contact on the head and/or on the body 820 enables an electrical connection for signal communication when the head 888 is screwed onto the respective component.

And it is noted that in at least some exemplary embodiments, while body 820 is presented as a separate component from body 810, in some exemplary embodiments, one or more of the features of the body 820 are part of the body 810. Indeed, in an exemplary embodiment, the structure that is identified as body 820 can instead be part of an integral body 810. To be clear, we are not disclosing that there is meaninglessness between the two bodies. All we are doing is describing that in the interest of textual and schematic economy, that one or more of the features of the body 820 could be present with the body 810. By way of example only and not by way of limitation, there may be body 820, and instead, body 810 encompasses everything that is shown with respect to body 820, except the gap that is shown between the two that is associated with the protrusion 813 (which would not be there if, for example, everything was part of a single component).

In any event, just briefly, in an exemplary embodiment, the combination of head 888 and body 820 can correspond to the second module, and this can operate as a plug type device, and analogous to a cork where the outer surface of body 820 and/or a seal on the outer surface of body 820 provides the sealing features when the body 820 is an passage 819, and then upon the removal of such, which can be executed by simply pulling head 888 away from body 810, and thus pulling body 820 out of passage 819, such establishes a passageway to enable the physical access from cavity 106 to cavity 199.

In an exemplary embodiment, one or more of the inner ear port devices according to the embodiments herein and/or variations thereof can be configured to wirelessly communicate with a component remote from the inner ear port device. By way of example only and not by way of limitation, as detailed above, in an exemplary embodiment, an RF inductance coil is utilized to communicate in a transcutaneous manner to a device that is an external device and/or otherwise located supercutaneously. In an exemplary embodiment, this can correspond to communicating to an RF inductance coil that is located in the ear canal by way of example. While embodiments of focused on the RF inductance coil technology, other embodiments can utilize other wireless communication regimes, such as, for example, infrared communication.

In an exemplary embodiment, the communication regime that is utilized is based on or otherwise can be the same as or can be a modified version of existing transcutaneous communication devices known in the art, such as those that are utilized for, for example, to establish communication between an implanted cochlear implant receiver stimulator and an external component of a cochlear implant. The communication regime can be that utilized by, for example, the device of FIG. 6C and/or 6D detailed above. The communication regime can be that utilized by, for example, the device of FIG. 4 above or the device of FIG. 3 detailed above with the device of FIG. 2 above. Again, this is all consistent with the concept as detailed above where some features of the arrangements of FIGS. 2 to 6D can be utilized to implement the teachings according to the inventions herein.

The communication regime (data and/or power) can be that corresponding to, for example, the Nucleus 6™ and/or Nucleus 7™ and/or Nucleus 8™ cochlear implants supplied by Cochlear LTD as is present or otherwise available in the United States of America and/or the United Kingdom and/or the Republic of France and/or the Federal Republic of Germany, and/or the Commonwealth of Australia and/or New Zealand and/or the People's Republic of China on Mar. 15, 2021. This can include telemetric aspects of the use communication regimes. Some embodiments can be limited to the communication of power to power the inner ear port device, while other embodiments can include the communication of data instead or along with power. And, as just noted, in some embodiments, the inner ear port device can be configured to provide telemetry to the external component. To be clear, in some embodiments, the RF inductance coil is in the aforementioned cochlear implants can be downsized or otherwise miniaturized at least with respect to the outer diameter thereof. In this regard, while the examples of the cochlear implants just noted often have an inductance coil that has an outer diameter of 30 mm, inductance coil's detailed herein are contemplated which have diameters of less than ½ to ⅓ or ¼ or 3/16 or ⅛ of those diameters. Also, embodiments include the utilization of the receiver and/or transmitter and/or transceiver components thereof, albeit utilized and potentially miniaturize for the utilization of the inner ear prostheses according to the embodiments herein.

Still, in at least some exemplary embodiments, the transcutaneous link can be utilized to power electronics of the port device. In an exemplary embodiment, the power is direct power, and the port device will not operate without the transcutaneous power link. In an exemplary embodiment, the power is in direct power, and the transcutaneous link is utilized to power a capacitor and/or a rechargeable battery or the like. And in these embodiments, the inner ear port device can be configured to operate the electronics in the absence of the external component providing power via the link.

With respect to the embodiments where the port device will not operate without the transcutaneous link, in an exemplary embodiment, the transcutaneous link can be utilized to activate, for example, a therapeutic substance release device that can enable the release of therapeutic substance by the port. This can also be utilized in a case where, for example, the sensors are to be located for a long period of time in the cochlea, the sensors are utilized only periodically. Accordingly, in an exemplary embodiment, there is a method of periodically providing a transcutaneous communication signal to the port device, such as, once a week or once a month or a few times a month or once every quarter, etc., which could be established by periodically placing a narrow communication device into the inner ear canal, which communication device is provided with an RF inductance coil to provide a signal to the implanted port. In an exemplary embodiment, it can be enough to simply provide a power signal to the port, where upon receipt of power, the port activates accordingly. Conversely, there can be logic circuitry or other types of circuitry in the port, which can receive data and analyze the data and operate accordingly, where the data can be controlled data. This is analogous to the regime that is utilized by the aforementioned cochlear implants communication regime. Accordingly, at least some exemplary embodiments of the port device include at least some of the structure/components/firmware/software, etc., of one or more of the implantable portions of the aforementioned cochlear implants noted above that can enable the communication in a utilitarian manner.

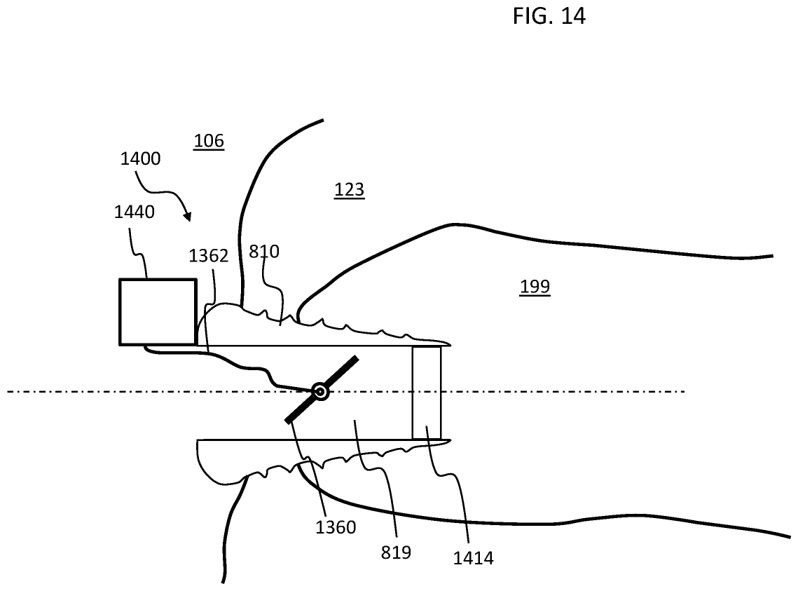

In an exemplary embodiment, the inner ear port device establishes a fluid valve between fluid of the inner ear and an outside of the inner ear. By way of example only and not by way of limitation, FIG. 14 depicts an exemplary embodiment of a device 1400 where, for example, a butterfly valve 1360 is positioned within the passageway 819. In an exemplary embodiment, the butterfly valve 1360 includes an actuator, which can include an electric motor or a MEMS component or a piezoelectric-based device, that, when actuated, moves the valve 1360 to open and/or close the passageway 819 to enable or prevent fluid communication between the cavity 199 and, for example, portions of the passageway 819 that are proximal the valve 1360. In an exemplary embodiment, the inner ear port device establishes a fluid valve between fluid of the inner ear and an outside of the inner ear.

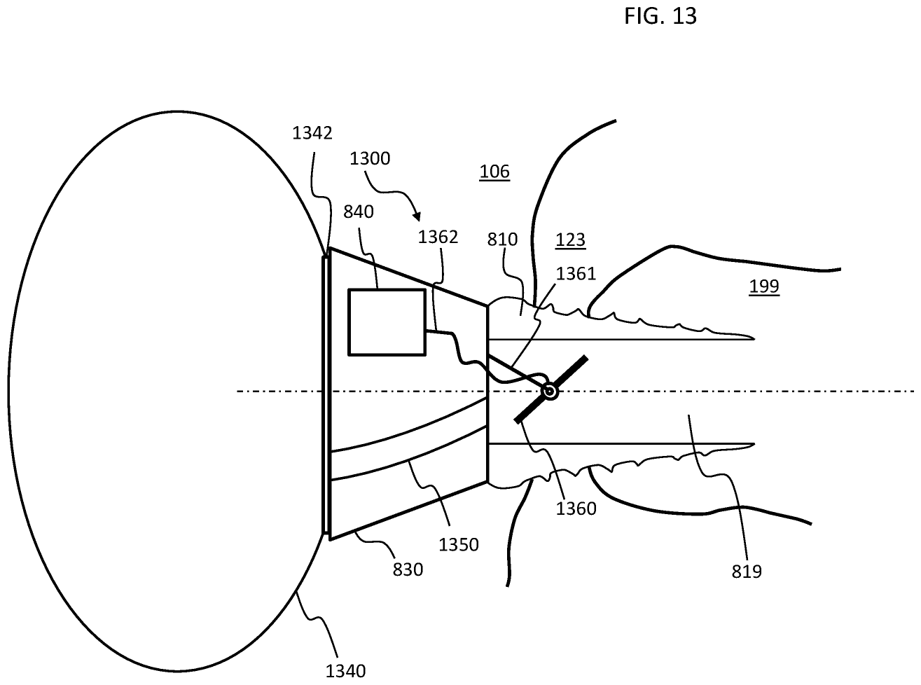

FIG. 13 presents an exemplary embodiment of a device 1300 where by way of example only and not by way of limitation, there is a therapeutic substance reservoir 1340, which can be, for example, a stretched elastomeric material in the form of a flexible container, which could have balloon-like qualities, where, the inside is over pressured relative to the pressure within the cochlea and/or within the middle ear, and thus the therapeutic substance contained therein, which can be a fluid (liquid and/or gas) thus has a predisposition to exit the reservoir 1340. The reservoir 1340 is releasably connected to the head 888 by coupling 1342 which can be configured to enable the removal and installation/attachment of the reservoir to the head 888, or can be permanently fixed thereto (the removable feature/releasably connection feature enables the reservoir 1340 to be replaced, such as after the reservoir is depleted, for example). Here, there can be a conduit 1350 that extends from the reservoir 1340, more accurately, from an opening in the reservoir, or otherwise a port in the reservoir, that enables fluid communication from the interior of the reservoir 1340 through the head 888 to the passageway 819. In this exemplary embodiment, when the valve 1360 is closed, the therapeutic substance, which is under pressure owing to the nature of the reservoir 1340, cannot travel from the proximal side of the valve to the distal side of the valve, and thus cannot enter the cavity 199, and thus cannot comingle with the perilymph therein. When the valve 1360 is open, therapeutic substance can travel into the cavity 199, and thus commingle with the perilymph therein. The valve 1360 is supported by strut 1361, which is connected to the head 888. In an exemplary embodiment, where the head is removable from the body 810, removal of the head will also bring the valve 1360 out as well, and thus the only part that would be permanently fixed is the body 810.

As can be seen, electrical leads 1362 extend to the valve 1360, and these electrical leads can be configured to enable electrical communication between a control unit 840 so as to control the valve 1360. In an exemplary embodiment, control unit 840 can include logic circuitry to control the actuation of the actuator of valve 1360 in a controlled manner. In an exemplary embodiment, this can be a predetermined algorithm which opens and closes the valve periodically according to a schedule. Alternatively, and/or in addition to this, control unit 840 can be linked to the sensor(s) (not shown), which sensors can sense a chemical composition and/or quantity or quality of substances within the perilymph and/or the perilymph itself, and then control the valve 1360 to meter the therapeutic substance into the cochlea 199. The control unit 840 can be a microprocessor or can be a chip based logic circuit. Instead of the controller 840 or in addition to this, in an exemplary embodiment, the valve 1360 can be linked to an RF inductance coil, and upon the RF inductance coil receiving power externally, the generated current in the coil can be utilized to actuate the valve 1360. In an exemplary embodiment, the valve 1360 can be spring loaded to the open or closed position. In this regard, in an exemplary embodiment, the unpowered state can be closed, and then when power from the external devices periodically delivered to the implanted port, the valve would open. In this sense, the valve could be a “dumb” valve that simply reacts to external power-it is akin to flipping a light switch—the implanted RF coil outputs electrical current to the motor or the actuator of the valve 1360, which actuates the actuator (just as a light goes on when the current is applied). Conversely, in an exemplary embodiment, the inner ear port device can be a “smart” device/implant. This can be achieved, by way of example only and not by way of limitation, via the control unit 840, which can be a logic circuit that is programmed to analyze data and control the valve accordingly. Again, as noted above, the data could be the properties of the perilymph or otherwise the substances within the perilymph. Upon a determination by the logic of the control unit 840 that, for example, a level of therapeutic substance, which can be measured by the sensors, falls below a certain level, the control unit 840 could open the valve, and maintain the valve, for example, until the level of therapeutic substance within the perilymph achieves a certain level. In an exemplary embodiment, the valve can be opened and then closed and opened and then closed with periodic intervals to gradually release amounts of the therapeutic substance. This could enable a stabilization period where the readings from the sensors can be more accurate (e.g., because the therapeutic substance would be given time to disperse). As seen above, in an exemplary embodiment, the battery or other power storage devices can be utilized to provide electrical charge to power one or more of the components that enable the implant to be a smart implant. Alternatively, and/or in addition to this, the RF communication system detailed herein can be utilized to provide electrical power to one or more of the components that enable the implant to be a smart implant. This power could be utilized to directly power those components and/or can be utilized to charge a rechargeable battery or power source. In at least some exemplary embodiments, this enables the implant to be utilized when the external power supply is not present or otherwise is disconnected or otherwise the transcutaneous communication link has been broken.

In an exemplary embodiment, the battery can be a permanent battery that is permanently part of the implanted component. In an embodiment, the battery can be replaceable. Indeed, in an exemplary embodiment, the battery is utilized so that it is depleted, either partially or fully, and then the techniques detailed herein can be utilized to replace that battery with a new battery.

Other embodiments can utilize other phenomena that can be sensed or otherwise detected by the inner ear port device to determine the actions of the inner ear port device in some exemplary embodiments of a smart implant (including a smart prosthesis).

FIG. 14 presents another exemplary embodiment of a smart implant 1400, where the body 810 supports a housing 1440 that hermetically encloses control circuitry that controls the valve 1360, while in other embodiments, housing 1440 instead simply houses an RF inductance coil where energizement thereof opens the valve 1360 in accordance with the teachings above. In this exemplary embodiment, the therapeutic substances are delivered in a more traditional manner by filling or otherwise inserting a gel into the middle ear cavity. The gel will flow to the valve naturally to the passage 819, and thus to the valve 1360. The valve can be controllably opened and closed, to enable the therapeutic substance to reach the distal side of the valve. Shown in FIG. 14 is a barrier 1414 that has a structure, such as by way of porosity, that enables the therapeutic substance to defuse or otherwise flow from the passage 819 into the cavity 199, but which prevents the perilymph in cavity 199 from flowing outward into passage 819. In an exemplary embodiment, element 1414 can be a membrane. In an exemplary embodiment, element 1414 can be a device where the porosity thereof can be changed or otherwise controlled. By way of example only and not by way of limitation, in an exemplary embodiment, an electric current can be provided to element 1414 that reduces the porosity thereof or otherwise establishes a porosity thereof when the current is applied, or vice versa. Any device, system, or method that can enable the transfer of therapeutic substance from passage 819 into the cavity and can prevent perilymph, or at least prevent medically significant amounts thereof from flowing the other direction, into passage 819, can be utilized in at least some exemplary embodiments. To be clear, as can be seen, the proximal end of the passage 819 is open to the middle ear cavity 106 so that the gel or the like that is inserted into the middle ear cavity can reach the valve 1360, and thus the barrier 1414.

In at least some exemplary embodiments, the inner ear port device is configured to enable flow of perilymph or other inner ear fluids from the inner ear out of the inner ear. In an exemplary embodiment, there can be utilitarian value with respect to having the perilymph, for example, mixed with a substance within the reservoir. Still further, in an exemplary embodiment, embodiments can include the exchange of perilymph within the cochlea with artificial perilymph located outside the cochlea, and thus perilymph in the cochlea can be removed, and thus the perilymph can be transferred from inside the cochlea to outside the cochlea through the inner ear port device. The aforementioned valves detailed herein can enable such in at least some exemplary embodiments.

Thus, as seen above, some exemplary embodiments of the inner ear port device includes a valve that controls the fluid flow from the outside of the inner ear in general, and the cochlea in particular to the inside of the inner ear in general, and the cochlea in particular, and/or the other way around. This valve may be controlled from outside the body (e.g. wirelessly through e.g. Bluetooth or similar technology or through light based power transfer, and/or through manual manipulation) or from inside the human body, such as by way of example, in a closed-loop configuration with biosensors providing the control input.

In an exemplary embodiment, the inner ear device is configured to control the delivery of therapeutic substance into the inner ear, such as by way of example only and not by way of limitation, starting and/or stopping the movement thereof into the inner ear. In an exemplary embodiment, this can be achieved by, for example, controlling the valve. In an exemplary embodiment, the valve can be the butterfly valve or flapper valve detailed above (any valve herein can be a butterfly valve or a flapper valve, and embodiments where one is disclosed corresponds to a disclosure of the other), while in other embodiments, other types of valves can be utilized, such as by way of example only and not by way of limitation, a sphincter valve and/or a check valve/one-way valve and/or a ball valve, etc. Any device system and/or method that can enable the control of transportation of the therapeutic substance, including the stopping and starting of the substance, can be utilized in at least some in some embodiments.

In an exemplary embodiment, the control of transportation of the therapeutic substance can be controlled by the wireless systems detailed herein. Alternatively, and/or in addition to this, the control can be controlled by the onboard componentry. In an exemplary embodiment, the inner ear port device is completely autonomous. In an exemplary embodiment, the inner ear port device is configured to operate in a manner analogous to the Predator Drone with respect to how the drone flies and obtains data by way of example. In this regard, the Predator Drone can be configured to selectively choose a utilitarian altitude and/or adjust control surfaces to execute the mission profile without input from a remote device. The drone can also be configured to determine locations and directions to point it is on board cameras (roughly analogous to sensors of the inner ear port device).

In view of the above, it is noted that embodiments include an inner ear device including one or more or all of the following: a wireless communication system (configured to send and/or receive, such as by the use of a receiver, transmitter and/or transceiver respectively), sensors that are configured to sense a physical phenomenon within the inner ear, logic circuits and/or electronics that can execute logic functions, including chips and/or processors.

In an exemplary embodiment, as differentiated from, for example, the arrangement of FIG. 5 and/or 4, which, as noted above, are not part of the invention, but provide teachings that can be utilized to implement some aspects of the invention, all of the componentry associated with the inner ear device is located within a volume of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 90, 100, 125, 150, 175, 200 or 300 or 400 or 600 or 800 or 1000 mm³ or any value or range of values therebetween in 1 mm³ increments. In an exemplary embodiment, the aforementioned volumes are established by a cube volume, or a volume established by rectangular sides (again, it is within-the device need not be a cube—this can be analogous to shipping volume specifications for a box or container) where the largest straight dimension of the side is 0.5, 0.6, 0.7, 0.8, 0.9, 1.0, 1.5, 2, 2.5, 3, 4, 5, 6, 7, 8, 9, 10, 15 or 20 mm or any value or range values therebetween in 0.1 mm increments.

In an exemplary embodiment, the entire device of the inner ear port is located within 0.5, 0.6, 0.7, 0.8, 0.9, 1.0, 1.5, 2, 2.5, 3, 4, 5, 6, 7, 8, 9, 10, 15, or 20 mm, or any value or range of values therebetween in 0.1 mm increments of a natural inner ear cavity.

In any event, returning back to the sensor embodiment, it can be seen that in some exemplary embodiments, there is an inner ear device, comprising, a tissue interface portion configured to be attached to tissue of and/or proximate an inner ear of a human. This tissue interface portion can be the body 810 as detailed above. The inner ear device further includes a sensor (one or more sensors). In this exemplary embodiment, the inner ear device is a passive implant configured to sense at least one phenomenon related to the inner ear of a human. In an exemplary embodiment, the sensor is a biosensor in contact with perilymph and is configured to measure or property thereof. In an exemplary embodiment, the biosensors can be placed into contact with perilymph to measure protein (electrodes for impedance based sensing), pH, temperature, pressure, gait, movement etc.

In an embodiment, the device is configured to enable the component to be removed from the tissue interface portion when the tissue interface portion is removably permanently fixed to the barrier establishing the inner ear of a human, and the component at least partially seals the passage and provides one or more passive features. As will be detailed below, the component can completely seal the passage. In other embodiments, the device is configured to enable the component to be removed from the tissue interface portion when the tissue interface portion is removably permanently fixed to the barrier establishing the inner ear of a human, and the device also includes a removable seal apparatus configured to unsealably seal the passage. In some embodiments, the device is configured, when the plug or cap (seal apparatus), etc., is removed from the device, so that the passageway is open when the component is releasably attached to the tissue interface portion and/or a portion of the device supported by the tissue interface portion. In some embodiments, the component is configured, when the plug/cap etc., is removed from the device to unsealably seal a local portion of the passageway when the component is releasably attached to the tissue interface portion and/or a portion of the device supported by the tissue interface portion. There can also be a passageway thought he component, which passageway can also be sealed by another plug or cap (there can be two plugs or caps, or can be only that sealing the passage of the component).

In an exemplary embodiment, the port device can include circuitry corresponding to the so-called lab on a chip (LOC) concept. In an exemplary embodiment, the port 810 and/or the component inserted therein are coupled thereto can have the LOC. The LOC can be in signal communication with the electrodes or the working end of the sensors so as to implement any one or more of the analysis detailed herein or otherwise having utilitarian value with respect to analyzing the various biomarkers detailed herein. In an exemplary embodiment, the electrodes, which can be platinum electrodes, can be coated with a substance where, if a given protein is present, the electrical properties associated with current flowing between one or more of the electrodes will change, thus indicating the presence of that protein. Concentrations of various biomarkers can be measured. Embodiments can include evaluating the concentrations, and thus altering the therapeutic substance delivered and/or the amount thereof that is delivered and/or the rate thereof that is delivered or otherwise increasing or decreasing a rate of testing. The sensors can be antihistamine biomarker sensors. The sensors can utilize a histamine pathway concept. The sensors can look for biomarkers that are markers for disease. Upon an identification that the biomarkers are present, a warning could be provided to the recipient and/or to a healthcare professional via a communication link such is any of those detailed herein with an external device. Periodically, data transfer could be executed from the implanted port apparatus in accordance with the teachings detailed herein. For example, the data resulting from the sensors and/or the analysis thereof by the port device could be stored in a memory chip which can be uploaded once every week or month or day for that matter, to an external device or to a lab device, such as a communication wand used by a healthcare professional.

In the above, it can be seen that in an exemplary embodiment, there can be an active first component of the port device, which active first component can be a sensor. The port device can be configured to evaluate the data obtained by the sensor and, based on the evaluation, provide a warning to a recipient of the port device, which warning would be automatic. In an exemplary embodiment, the port device could have an actuator or some form of device that is configured to evoke a hearing percept however minimally, thus providing the warning. For example, the warning could be a low-frequency or a high frequency series of pulses, which would reoccur for a short period of time statistically likely to be noticed by the recipient but not occurring for such a long amount of time so as to be a nuisance.

Still, in some embodiments, the warning would be transmitted over the transcutaneous link to an external device. Again, periodically, the recipient could utilize a handheld device to download data from the port device.

In an exemplary embodiment, as noted above, this can be utilized as a basis for which to automatically control the delivery of therapeutic substance into the cochlea. In an exemplary embodiment, this can be utilized simply to provide data to a healthcare professional so that he or she can evaluate the status of an inner ear condition and/or notify the person/recipient of a pending medical episode, such as, for example, a pending vertigo attack, etc.

In an exemplary embodiment, the implants detailed herein can utilize these sensed at least one phenomenon related to the inner ear to determine a balance and/or condition of the human at a level beyond hearing-related features. By way of example only and not by way of limitation, properties of the fluid within the semicircular ducts (one or more of these ducts) are monitored utilizing the sensors of the inner ear prostheses to evaluate the balance state of the recipient.

In at least some exemplary embodiments, the sensor of the inner ear device can be a movement sensor, and the inner ear device can be part of a balance prosthesis/balance system.

Consistent with the teachings detailed above, the tissue interface portion can be configured to be permanently fixed to tissue, such as the bone that establishes the boundary between the middle ear in the inner ear, or more accurately, such as the passageway that can be established therethrough. In an exemplary embodiment, this inner ear device can include an on board power source that provides power to the sensor. This can be any of the aforementioned power sources, such as, for example, a battery or a capacitor. The power source can be rechargeable or otherwise can be a long-term power source that is replaced after a number of years by way of example.

Still further, consistent with the teachings detailed above, this inner ear device can include a wireless transcutaneous communication system configured to receive power to power the sensor and/or transmit data based on data obtained by the sensor. This can be an RF communication system, or can be another type of wireless system, such as, for example, an infrared system. In an exemplary embodiment, the sensor can be in electrical communication with an RF inductance coil and/or with an infrared signal generator, and the phenomenon sensed by the sensor can result in the center outputting a signal that can cause the RF inductance communication system and/or the infrared signal generator to provide output that can be captured by an external component or some other component (the communication system need not be a transcutaneous communication system in this embodiment, while in other embodiments it is such-here, there can be a receiver located remote from the prostheses but still in wireless communication range with the implant), where the other component or the external component can capture the output. The captured output can be evaluated. By way of example only and not by way of limitation, the sensor could output a signal that causes the infrared generator of the infrared communication system to shift a wavelength, which shift represents data which can be analyzed. Still, in some embodiments, there can be a signal booster or the like or a device that creates residents, such as which of the utilitarian with respect to the embodiment where the wireless communication is an RF inductance coil.

And it is briefly noted that while RF inductance coils are often the focus of the communications systems detailed herein, other RF systems can utilize other types of antennas, such as monopole and/or dipole antennas or otherwise antennas that extend in a more linear fashion than the RF inductance coils that are utilized in the aforementioned cochlear implants.

A wireless communication chip can be located in the head 888 or at another location of the port device, which chip can be, for example, a Bluetooth communication chip and/or a similar communication regime chip that can enable communication in general, and, in at least some exemplary embodiments, data communication. In an exemplary embodiment, the communication system is capable of enabling communication with a mobile phone and/or a smart phone and/or or other external device within one, two, three, four and/or five feet or more of the chip. Accordingly, embodiments include utilizing the smart phone, for example, to obtain data from the port device and/or to provide data to the port device. In an exemplary embodiment, the data can be data that is utilized to initiate or otherwise “program” one or more of the smart components detailed herein.

The power and the data transfer protocols can be those utilized by one or more of the aforementioned medical devices in the form of cochlear implants and/or the devices detailed above in FIGS. 2 to 6D, by way of example only and not by way of limitation.

As noted above, embodiments can enable repeated sealingly access from the middle ear to the inner ear through a sealable passage in the implant. This can be achieved, by way of example, via the head 888 that can be screwed onto the body 810 as detailed above. Alternatively, and/or in addition to this, in an exemplary embodiment, this can be enabled by, for example, the aforementioned flapper valve of the embodiment of the implant 1400 detailed above.

FIG. 10 depicts an exemplary implant/device 1000, that includes electrodes 844 with respective leads that are supported by body 1030, that can be made of PEEK any thermoset, thermoplastic or elastomer that can enable the teachings herein. As can be seen, implant 1000 includes a funnel 1023 at the proximal end of element 820. This can have utilitarian value with respect to guiding a termination of a syringe to the passage through element 820 and/or the device 1000 when inserted into body 1010. In an exemplary embodiment, the leads extend to a device that is located in the outer ear (more on this in a moment). It is noted that other embodiments, the leads are attached to a control unit attached to body 1030. Here, the electrodes are established by the ends of the leads 842.

FIG. 11 presents another exemplary embodiment of a relatively large support body 1130, that can be made of the material of body 1030. Here, the body 1130 is located in the tubular body 1011. The tubular body 1011 has in the passageway therethrough, a spring ring 1122 located therein, which prevents distal (further distal) movement of the body 1130. In an alternate embodiment, spring ring 1120 could instead be a ring screwed into the inside of the tube 1011. In this regard, the inside of tube 1011 could be threaded. In this regard, in some embodiments, the body 1130 is threaded into the tube 1011. The body 1130 may or may not have threads—the threaded coupling can be established by the flexible nature of the body 1130. As seen, the proximal portion of the body 1130 is proud of the proximal portion of the body 1011. This can have utilitarian value with respect to enabling removal and/or replacement of the body 1130, such as can be the case in the event of wear and/or age of the body 1130. It is also noted that in an exemplary embodiment, element 1130 could instead be a plug. The plug can be removed to enable access into the inner ear cavity 199. The plug can be a rigid plug or operate in the form of a cork. Further, the proximal end of the plug 1130 or body for that matter could also have a wider portion otherwise could be such that the overall structure is T-shaped, to enable a larger area to be gripped in the event of removal.

Embodiments of the plugs and/or other devices that “fill” the body that interfaces with the tissue of the inner ear detailed herein, or the body itself for that matter, are configured to avoid leakage of fluid from within the cochlea or otherwise within the inner ear to the middle ear or otherwise outside the inner ear, or at least avoid substantial leakage that would have a noticeable deleterious effect and/or an annoyance effect. By way of example only and not by way of limitation, a leakage rate can be limited to (no more than) 0.1 to 10 microliters or any value or range of values therbetween in 0.01 microliter increments. These can be absolute values, or values that occur after a period of time lasting 1, 2, 3, 4, 5, 6, 7, 8, 9 or 10 days or weeks or any value or range of values therebetween in 1 hour increments.

While some embodiments detailed above have been directed towards an inner ear device that includes a wireless communication system, it is noted that other embodiments can alternatively and/or in addition to this include a communication system that can be configured to interface with a wired receiver and/or transmitter. By way of example only and not by way of limitation, a communications jack receptacle can be located in the head or the like, where, via a surgical procedure, a jack from a transmitter and/or receiver can be placed into the communications jack, to establish communication. This can be utilized to upload or download data, or to control the middle ear device albeit during a temporary period where the implant is in communication with this transmitter and/or receiver. Still further, the wired communication can be permanent or semipermanent, and can lead to another component that is more closely located to the outer skin of the recipient. By way of example only and not by way of limitation, electrical leads can extend from the body or the head or some other component of the inner ear device to a location, for example, proximate the tympanic membrane and/or at a location within the skin that is proximate the ear canal. Wireless transcutaneous communication could be utilized to achieve the communication over the remaining distance of the external component.

Thus, in some embodiments, the inner ear port device is made of a single unit, while in other embodiments, the inner ear port device is made of multiple units. The inner ear port device can be configured to be secured into the labyrinth (cochlea, semi-circular canals, and/or otolith, depending on the embodiment), and can be utilized to provide direct access to inner ear fluid and/or tissue (perilymph, endolymph, etc.). In some exemplary embodiments, the enablement of the direct access to inner ear fluid can enable measurements of biomarkers in inner ear fluid, can enable delivery of drugs and/or other substances, including implants into inner ear fluid, and/or can enable sampling of inner ear fluid to allow for analysis inside the port and/or outside the body. One implementation of the inner ear port, as seen above, includes two units, where one is configured to be secured into and/or to bone or tissue and the other is configured to be attached to and/or inserted into the unit secured into bone or tissue.

Thus, in at least some exemplary embodiments, there is an inner ear device, comprising a tissue interface portion configured to attach to tissue of and/or proximate an inner ear of a human and provide a passage from outside the inner ear to inside the inner ear. This can correspond to, for example, body 810 alone or the combination of the components detailed above with respect to some of the embodiments. In an exemplary embodiment, the device further includes a container releasably attached to the tissue interface portion and/or a portion supported by the tissue interface portion (e.g., body 820 if body 820 is used in combination with body 810, or head 888, if head is used in combination with body 810-of course, if head 888 is part of body 810, then that is the “releasable attached to the tissue interface portion). In this exemplary embodiment, the container is configured to contain a therapeutic substance, and in some embodiments, the container contains the therapeutic substance.

Here, the inner ear device is configured to control itself and/or to be controlled remotely to deliver therapeutic substance contained in the container to an inner ear. This can be done, for example, with respect to the former, utilizing the onboard logic circuitry (where, the inner ear device is configured to control itself to deliver therapeutic substances contained in the container to the inner ear), and with respect to the latter, utilizing the wireless communication regimes detailed above. In at least some exemplary embodiments, the container is configured to be located entirely within a middle ear cavity of a human.

In an exemplary embodiment, the inner ear device has no stimulative capabilities. In an exemplary embodiment, the inner ear device has no componentry configured to electrically stimulate tissue to evoke a sensory response. In an exemplary embodiment, the inner ear prostheses has no componentry configured to mechanically stimulate tissue to evoke a sensory response. The device can have in some embodiments, the above-noted sensors, which could include electrodes which would provide an electrical current as noted above. However, providing that this does not electrically stimulate tissue to evoke a sensory response, such would meet the aforementioned qualification that the prostheses has no componentry configured to electrically stimulate tissue to evoke a sensory response.

In some embodiments, the inner ear device is configured to release the therapeutic substance contained in the container through active transportation. Thus, here, it can be seen that the implant can include a drug or other therapeutic substance reservoir that can be replaced (and/or refilled—more on this in a moment). The drug or other therapeutic substance can be released into the inner ear though active mechanisms or regimes. The drug or other therapeutic substance influx can be regulated by way of example only and not by way of limitation, utilizing valves and/or the pressure alone and/or in combination with inlet holes/pores, stretchable membranes, etc. Thus, in some embodiments, the inner ear device is configured to release the therapeutic substance contained in the container through active transportation. Further, in some exemplary embodiments, the inner ear device is configured to self-regulate release of the therapeutic substance contained in the container into the inner ear. As seen above, this can be accomplished via a feedback loop in conjunction with the sensors or otherwise by logic circuitry, etc. This is as distinguished from, for example, the inner ear device being controlled by an external component or an inner ear device that delivers a therapeutic substance without self-regulation, both of which are also included in at least some exemplary embodiments. Of course, the two are not mutually exclusive; some embodiments can have both self-regulation and the ability to be controlled externally.

As noted above, in some exemplary embodiments, the drug reservoir is releasable from the tissue interface component (and/or a component between the tissue interface component, such as the head 888). In other embodiments, the drug reservoir is permanently attached. In an exemplary embodiment where the drug reservoir is permanently attached, the drug reservoir can be refilled or otherwise resupplied. In an exemplary embodiment, this can entail conveying a needle/termination of a syringe into the middle ear, and then passing through a receptacle, and then refilling the reservoir. In another exemplary embodiment, by way of example only and not by way of limitation, a refilling port can be located on the reservoir or in fluid communication there with, and a refueling tube or some other device can be attached to the port, and the therapeutic substance can be delivered therethrough. In an exemplary embodiment, the reservoirs are sized and dimensioned or otherwise configured so that a total load of the therapeutic substance, in a scenario where all of the therapeutic substance was released in a relatively short time, such as all the once, in the event of an accident, or otherwise in the event of a failure mode, would not result in a toxic level and/or an above toxic level of therapeutic substance being released into the person. Alternatively and/or in addition this, multiple reservoirs could be utilized that have these features individually, where the likelihood of a series of failures were therapeutic substances into or more reservoir is being released at the same time otherwise in close proximity is unlikely as a matter of statistics.

It is noted that while the above embodiment associated with refilling or otherwise resupplying the container was described with a container that is fixed and otherwise not releasably attached to the tissue interface component or whatever component is an issue, in other embodiments, one or more of the above features associated with refilling can be applicable to the container that is releasably attached.

It is further noted that the phrase “releasably attached” refers to a structure that enables the container to be readily detached in a normal and expected manner so as to permit resupplied. This is as distinguished from, for example, the mere ability to disassemble various components. That is, even if, for example, the container could be saved for example, cut from the tissue interface, such would not correspond to releasably attached.

And further to the embodiment of FIG. 13, in an embodiment, the inner ear device can include a fluid valve between fluid and the inner ear and an outside of the inner ear. The valve is shown in FIG. 13 as valve 1360. In at least some exemplary embodiments, the implant is configured so that the valve can be adjusted to control an amount of therapeutic substance to be released into the inner ear from the container. The valve can be controlled in any one or more of the aforementioned ways to accomplish any one or more of the aforementioned method actions. The valve can be utilized in conjunction with other control functionalities, such as, for example, regulating the pressure within the reservoir 1340 and/or the pressure behind the valve 1360.

In an exemplary embodiment, the valve or other therapeutic substance delivery regulation means is configured to and/or controlled to prevent deleterious and/or annoying pressure fluctuations within the inner ear. In an exemplary embodiment, the valve or other therapeutic substance delivery regulation means is configured to and/or controlled to maintain a level of pressure within a certain boundary, limit any pressure fluctuations to within a certain range. In an exemplary embodiment, the actions detailed herein are executed and the devices and systems enable the execution of a therapeutic substance delivery or any other actions detailed herein in some embodiments, into the inner ear that maintains the pressure within the inner ear within a 5, 6, 7, 8, 9, 10, 11, 12, 13, 14 or 15% variance from a mean, median, and/or mode and/or from a baseline pressure (the pressure just before the start of the action). In an exemplary embodiment, the pressure regulation is achieved by active means, such as, for example, a sensor that senses the pressure and thus will open the valve upon the attainment of a certain pressure read by the sensor, for example.

Consistent with the teachings detailed above, in some exemplary embodiments, the tissue interface is located in bone establishing a barrier between the middle ear and the inner ear. In some exemplary embodiments, the tissue interface has been implanted in the bone for at least and/or equal to 3, 4, 5, 6, 7, 8, 9, 10, 11 months or 1, 2, 3, 4, 5, 6, 7, 8, 9, 10, 11, 12, 13, 14, 15, 16, 17, 18, 19, or 20 or more years, or any value or range of values therebetween in one week increments.

Some embodiments include the action of utilizing the inner ear port device as a cochleostomy without a sheath for insertion of a cochlear implant electrode array.

In view of the above, it can be seen that in an exemplary embodiment, the device that includes a tissue interface portion also includes a powered sensor, and the device is a non-stimulative device configured to sense at least one phenomenon related to the inner ear of the human. In an exemplary embodiment of this exemplary embodiment, as detailed with respect to FIGS. 19 and 20, the powered sensor is part of the tissue interface portion, and the tissue interface portion has a passage configured to enable access into a cochlea, wherein the passage is unpluggably plugged to seal the passage.

Some embodiments include an inner ear device that is a device that is dedicated to the functionality of establishing long-term biocompatible ready access to the inner ear from the middle ear. This as distinguished from, for example, a cochlear implant, where a portion of the implant extends from the middle ear into the inner ear. Such a device is configured to provide sensory stimulation. In some exemplary embodiments, the inner ear device has no componentry configured to electrically and/or mechanically stimulate tissue. In an exemplary embodiment, the inner ear device has no componentry configured to evoke a sensory response of the human. In an exemplary embodiment, the inner ear device has no componentry configured to electrically and/or mechanically stimulate tissue and/or fluid to evoke a sensory response of the human. This is distinguished from, for example, the potential that one or more of the electrical devices detailed herein may apply current somehow to tissue of the recipient. Further, in some embodiments, even if there is stimulation to tissue, providing that does not evoke a sensory response of the human, such would still be within the scope of some embodiments. To be clear, in some embodiments, there is no arrangements of the inner ear device that is stimulative. In some embodiments, the purpose of the implant is to provide the long-term ability to access the inner ear from the middle ear.

Thus, in view of the above, it can be seen that in some embodiments, there is a device, comprising a tissue interface portion configured for securement to tissue of and/or proximate an inner ear of a human and provide a long term passage from outside the inner ear to inside the inner ear, and a therapeutic substance delivery device at least indirectly releasably attached to the tissue interface portion and/or a portion of the device supported by the tissue interface portion, wherein the device is configured to actively control itself and/or to be actively controlled remotely to deliver the therapeutic substance to an inner ear, and the therapeutic substance is configured to be located entirely within a middle ear cavity and/or the inner ear of a human.

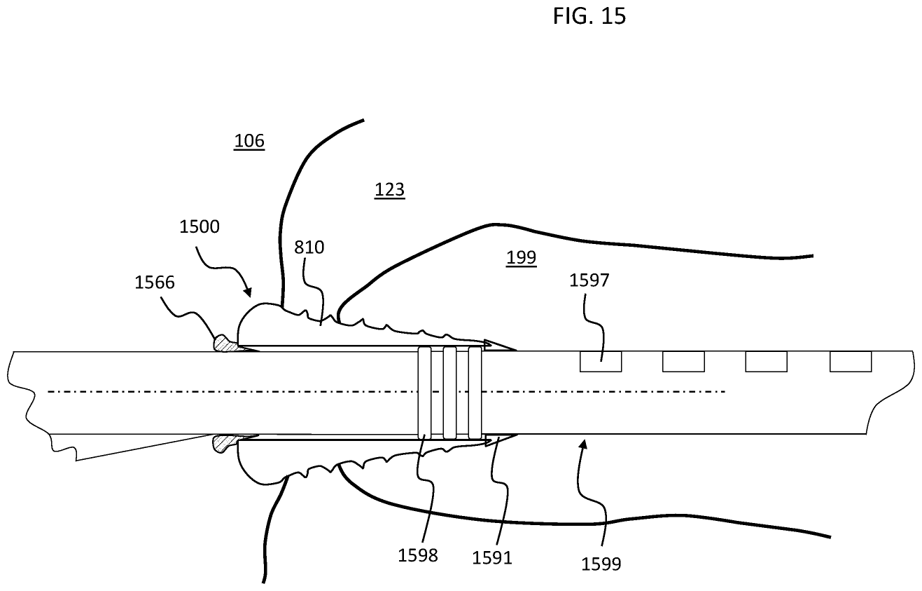

But note that some embodiments can utilize the inner ear device to access the inner ear for the purposes of inserting a device that is configured to stimulate the cochlea. By way of example only and not by way of limitation, FIG. 15 presents an exemplary embodiment of an inner ear port 1500. Here, the port 1500 includes the body 810, which can correspond to the above embodiment. In this arrangement, a cochlear implant electrode array 1599 extends through the inner ear port device 1500. Note that the cochlear implant electrode array 1599 is not part of the inner ear port device 1500. Instead, it is utilized in conjunction therewith. Accordingly, what is shown in FIG. 15 is a system that includes an inner ear port device 1500 and a cochlear implant electrode array (in fact, because the cochlear implant electrode array is attached to the receiver stimulator of the cochlear implant, what is shown in FIG. 15 is also a system that includes the inner ear port device 1500 and an implantable portion of a cochlear implant). In an exemplary embodiment, the arrangement of FIG. 15 can be achieved by executing that method where an active component and/or some other component is removed from contact with body 810, which component closes the passage through the body 810, and that removed component is replaced with the cochlear implant electrode array as shown. In this exemplary embodiment, the cochlear implant electrode array 1599 includes a plurality of stimulating electrodes 1597, some of which are shown (the full extent of the array is not shown for purposes of schematic economy). Also shown are ribs 1598. These ribs abut the inner surface of the passageway through body 810, and can establish a fluid tight seal. In an exemplary embodiment, the electrode array 1599 is specially designed to work with the inner ear port device 1500. In an exemplary embodiment, a “wedge seal” 1565 is part of the electrode array 1599. As the electrode array is inserted into the cochlea through the passageway in the body 810 the pertinent distance, the wedge seal 1566 enters the passageway through the body 810, and wedges itself therein, thus establishing a second seal in addition to the ribs 1598. The above said, in an exemplary embodiment, a separate seal instead and/or in addition to this can be placed at the interface. In an exemplary embodiment, a form-in-place seal can be applied after the electrode array 1599 is inserted into the cochlea. That said, the locations of the electrode array that can be estimated to be proximate the inner surface of the body can be covered with the form-in-place seal material such that when the electrode array is inserted through the passage, the sealing material which has been carried into the passage by the electrode array 1599 contacts the inner surface of the body 810, and thus establishes a seal after a modicum of curing.

In an exemplary embodiment, the electrode array 1599 has components that prevents further insertion into the cochlea and/or prevent movement of the electrode array 1599 in the opposite direction, or otherwise frustrate such movements. By way of example only and not by way of limitation, as can be seen in FIG. 15, there is a wedge seal 1591 that extends about the outer periphery of the intracochlear portion of the electrode array 1599. In an exemplary embodiment, after the election array is inserted a sufficient distance, and the compressible material of the wedge seal 1591 (the compressible material could be silicone for example) clears the distal and of the passageway of the body 810, thus permitting the compressed wedge seal 1591 to “spring” outward again, thus pass the inner diameter of the inner passage, a slight pullback on the electrode array can jam the relatively sharp edges (in this exemplary embodiment) of the body 810 into the wedge seal 1591 is shown, thus creating an additional seal. But even without this additional sealing, this exemplary embodiment permits the electrode array 1599 to be held relatively stationary or otherwise prevents or otherwise frustrates rearward movement of the electrode array out of the cochlea. In an exemplary embodiment, seal 1566 is added after the election array is so positioned, and the seal 1566 can bond or otherwise correct the body of the extra cochlear portion of the electrode array, thus preventing the electrode array from moving forward or otherwise frustrating forward movement, and, in this embodiment, providing for additional sealing. Note also that in an exemplary embodiment, the surface of the passageway through the body 810 could be roughened so as to increase the friction forces against the ribs 1598. And in some embodiments, instead of the ribs and/or in addition to the ribs, the overall outer profile of the electrode array could be larger than the inner diameter of the passageway the body 810, so as to provide a friction fit and/or an interference fit to frustrate movement of the electrode array in the various directions. Also, such a fitting regimes can also create a seal, at least in embodiments where, for example, the electrode array is made of silicone at the interfacing portion.

An exemplary embodiment includes a minimally-invasive implantation method for a cochlear implant electrode array, which electrode array results in the ability to provide electrical stimulation to ganglion cells responsible for sensing higher frequency sounds. This method also includes the utilization of components, such as, for example, a grommet that is adapted for insertion into a cochleostomy formed in bony tissue adjacent the round window by way of example. The grommet can have an actual passageway through the center thereof. The array can be configured to be inserted through the actual passageway of the grommet, and methods include doing so. The electrode array and/or the grommet has a cross-sectional size that enables the array to snugly engage and otherwise fill the axial passageway when the electrode array is inserted to the prescribed depth. This snug fit prevents fluids, such as perilymph, from not passing through the axial passageway when the electrode array is inserted in the axial passageway. In some exemplary embodiments, there is a grommet, which can have any one or more of the features detailed above associated with the body 810, comprises a conical shape member having threads on an outer surface thereof, and a slot on a backside thereof. This lot can be configured to receive a flat head of a screwdriver like to enable the grommet to be screwed into the bone, whether there be a passageway there or via the use of self tapping features. Alternatively, and/or in addition to this, a hex head can be utilized to enable a wrench to be utilized to apply the torque to the grommet. In an exemplary embodiment, a rotational driving force applied to the slot (or hex head) on the backside of the grommet causes the grommet to be screwed into bony tissue surrounding the cochleostomy and/or causing the grommet to be screwed into unopened bone in the case of the self tapping grommet.

In an exemplary embodiment, there is a tool that enables the grommet or otherwise the body 810 to be supported at the end of the tool, and remain coupled to the tool in a manner that enables the tool to be utilized to transport the grommet to the location of insertion into the passage into the cochlea or for placement of the grommet against the bone so that the grommet can be utilized to self tap a hole into the cochlea.

It is noted that any one or more of the features described above associated with the grommet can be present in the body 810, such as, for example the flats or the hex head. It is also noted that while the grommet detailed above has been described in terms of utilization with a cochlear implant electrode array, in some other embodiments, any one or more of the other second modules detailed herein can be utilized with the grommet.

While the embodiment detailed above is focused on an electrode array of a cochlear implant, in an alternate embodiment, the component that is inserted through the body 810 or other device that establishes the inner ear port device can be a direct acoustic stimulator. Alternatively, and/or in addition to this, electrodes that treat tinnitus and/or balance or some other device that treats tinnitus and/or balance, such as a mechanical actuating device, can be inserted through the port device. And this can include embodiments where the port device is utilized to provide access to the interior of the semicircular canals and/or the vestibule (and is thus mounted on and/or through the walls of such). Still, at least some exemplary embodiments are directed to providing a port device that enables access at the scala tympani side of the cochlea.

It is noted that in some exemplary embodiments, a plurality of the inner ear port device can be utilized. In an exemplary embodiment, there could be a port device that is dedicated for the use of the cochlear implant, and then another one that is utilized for therapeutic substance delivery, or sensing, etc. In an exemplary scenario, a first inner ear port device is implanted into a human, and it is utilized for monitoring the inner ear of the human. Upon a determination based on the monitoring that there can be utilitarian value with respect to providing a therapeutic substance, one or more of the therapeutic substances detailed herein are provided, such as, by way of example, by attaching a reservoir or a therapeutic substance containing component to the inner ear port device. This could entail removal of the sensor module of the device and replacing such with the reservoir module/therapeutic substance delivery module, which could include components to enable the continued sensing function of the now removed sensor module. Alternatively, this could entail opening a passageway to the inner ear and attaching the therapeutic substance delivery module in addition to the existing sensing components of the prostheses. The therapeutic substance can be delivered as utilitarian. Then, at some point in the future, the person's hearing degrades, despite the application of the therapeutic substance for example, and thus a cochlear implant is deemed to be utilitarian. The sensing module or the therapeutic substance delivery module or both are removed in some embodiments, and a cochlear implant electrode array is provided through the port device. Because there is still utilitarian value with the specimen to sensing and/or providing therapeutic substance, a second inner ear port device is added at a location away from the first inner ear port device, and thus the functionality of the first inner ear port device is achieved by the second inner ear port device.

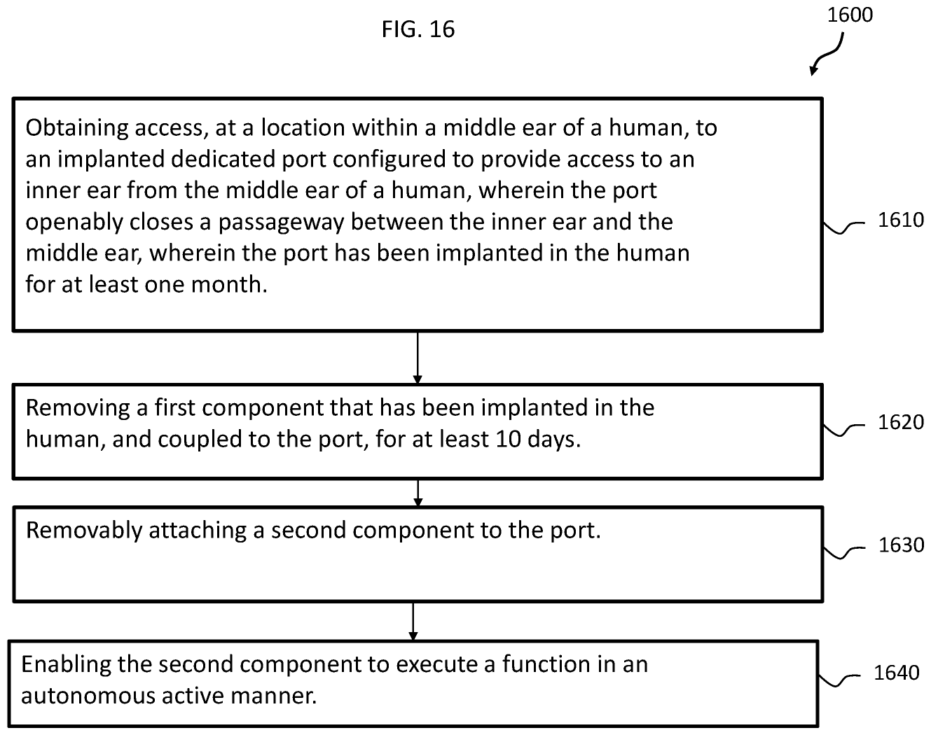

At least some embodiments includes methods. By way of example, FIG. 16 presents an exemplary algorithm for an exemplary method, method 1600 according to an exemplary embodiment. Method 1600 includes method action 1610, which includes the action of obtaining access, at a location within a middle ear of a human, to an implanted dedicated port configured to provide access to an inner ear from the middle ear of a human, wherein the port openably closes a passageway between the inner ear and the middle ear, wherein the port has been implanted in the human for at least one month, and in some embodiments, the port has been implanted in the human for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days or weeks or months or years or any value or range of values therebetween in one day/week/month/year increments.

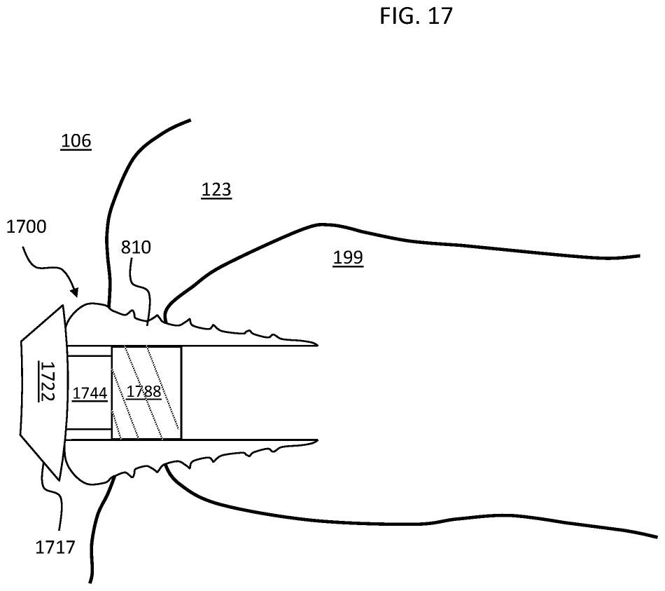

The port can be any of the port devices detailed herein and/or variations thereof, providing that they enable the openably closable feature. This is as distinguished from, for example, the naked body 810 shown in FIG. 15 without the electrode array. That said, as shown in FIG. 17, an exemplary embodiment of this port according to method action 1610 is seen with the cap 1717. Thus, in an exemplary embodiment of executing method action 1610, the port device 1700 is the port that is the subject of the method action. In an exemplary embodiment of this method, the method includes removing the cap from the port device, thereby obtaining access to the passageway into the inner ear.

In exemplary embodiment, the cap 1717 is screwed into the passageway, and thus there are mating threads within the passageway-the cap is made of an elastomeric material which also establishes a seal between the threads and/or at the head-in an exemplary embodiment, the cap is a composite component where the head is made of an elastomeric material, and the threaded body is made of titanium. FIG. 17 shows an example of this cap 1717, where the head 1722 is pulled against the proximal surface of the body 810, thus establishing a seal, as a result of the threaded body being screwed into the passageway towards the distal end thereof (the threaded body is 1788-non-threaded cylinder 1744 links the head to the threaded body 1788). In an exemplary embodiment, such as that shown in FIG. 17, the tension established by the continued threading of the cap 1717 into the passageway causes the head 1722 to flex as shown, further reinforcing the sealing feature-the natural/relaxed state of the head 1722 is a rectangular shape. In a further exemplary embodiment, ribs or a protrusion can be located on the proximal facing surface of the body 810, which can “cut” into the elastomeric material of the head 1722. But to be clear, in some other embodiments, the threaded body 1788 can also be made of the elastomeric material, where the interference between the rigid threads within the passageway through body 810 and the elastomeric threads establish a seal. Here, the cap 1717 is the mode of sealing. However, in an alternate embodiment, the body 1788 can be the mode of sealing (where body 1788 would correspond to a plug). In some embodiments, both a cap function and a plug function can be achieved via the seal apparatus.

Returning back to the method 1600. In an exemplary embodiment of method 1600, the port that is implanted has been implanted in the human for at least a month, while in some embodiments, the port has been implanted for any one or more of the aforementioned time periods noted above.

In view of the above, it can be seen that method 1600 further includes method action 1620, which includes removing a first component that has been implanted in the human, and coupled to the port, for at least 10 days, and in some embodiments, the first component has been implanted in the human for at least any of the above time periods. In an exemplary embodiment, this component could be the above-noted electrode array. In an exemplary embodiment, this component could be a therapeutic drug delivery pump that provides therapeutic substances. In an exemplary embodiment, this component can be any of the components detailed herein that are attached to the port that is directly attached to the tissue.

Method 1600 further includes method action 1630, which includes the action of removably attaching a second component to the port. This can be any of the components detailed herein that are attached to the port that is directly attached to the tissue. In an exemplary embodiment, this can be identical in design to the first component that is removed method action 1620, and thus an identical replacement component. By way of example, this can be a therapeutic substance delivery device that has a full reservoir for example, the replacement of which addresses the fact that the reservoir the first component has been depleted or is near depletion. In an exemplary embodiment, this second component could be the above-noted electrode array, where, for example, the first component was a therapeutic substance delivery device. In an exemplary embodiment according to such, it could be that an initial surgical operation is executed so as to provide a therapeutic substance delivery device in the recipient that provides therapeutic substance to the cochlea is an effort to preserve the cilia for example. Here, there is a decent likelihood that the recipient's hearing will eventually fail owing to degradation of the cilia, but there is also a decent likelihood that the delivery of the therapeutic substance may prevent such, at least in the short term. Accordingly, there is utilitarian value with respect to implanting the long-term/permanent port. The port serves the initial function of providing a support/attachment device for the therapeutic substance delivery device, and then serves the secondary function (not primacy, but temporally) of supporting the electrode array at a time when the cilia are sufficiently damaged or otherwise degraded that a cochlear implant is utilitarian.

In any event, method 1600 further includes method action 1640, which includes enabling the second component to execute a function in an autonomous active manner. This can be the delivery of therapeutic substance in an active manner, this could be the active sensing detailed herein, this could be the active establishment of a hearing percept via electrical stimulation resulting from a cochlear implant. The autonomous feature is that, once enabled, the device operates on its own in a manner concomitant with the autonomous teachings detailed above.

It is briefly noted that while the embodiments herein are typically directed to a port that accesses the ducts of the cochlea, in other embodiments, the port is utilized to access the interior of the semicircular canals. Accordingly, any disclosure herein relating to access to the ducts of the cochlea corresponds to a disclosure of accessing the interior of the semicircular canals for the purposes of textual economy unless otherwise noted, providing that the art enables such.

Embodiments where the function that is executed in an autonomous active manner with respect to method action 1640 can include the action of transferring a substance, foreign to the human, from outside the inner ear into the inner ear through the port, via an attached mating component. This can be executed by, for example, with respect to the former, transferring therapeutic substance from a reservoir into the inner ear.

In an exemplary embodiment, there are method actions that include, and there are devices and/or systems that enable, repeated access to the inner ear, such as to the cochlea, or the vestibule ducts, more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80 90 or 100 times or more or any value or range of values therebetween in one increment in a time period spanning 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months or years or any value or range of values therebetween in one month increments.

Embodiments include devices and systems that enable, and methods of, accessing perilymph and/or other fluids, directly (as opposed to indirectly) of the inner ear repeatedly in a safe manner, along a path or route that corresponds to that which was previously the case to do so, in some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 70, 80 90 or 100 times or more, or any value or range of values therebetween in 1 increments.

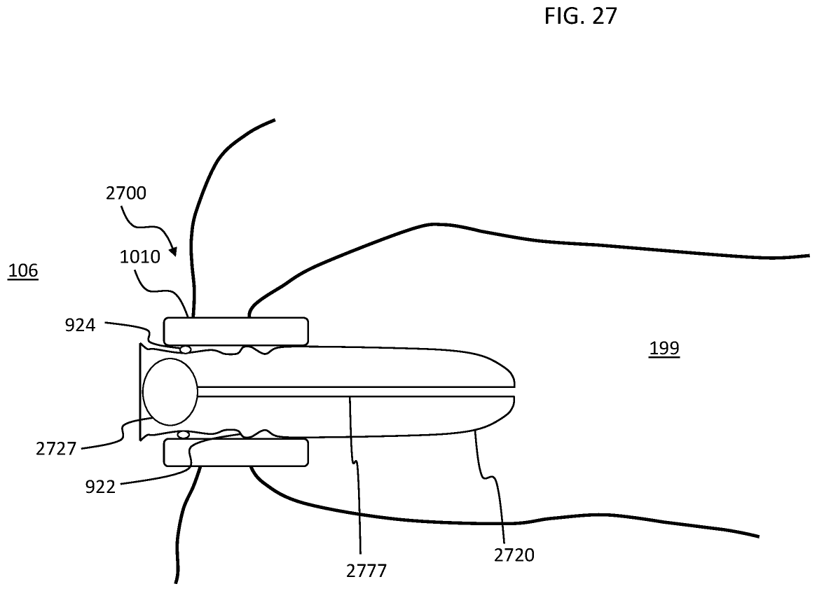

In this regard, FIG. 27 presents an exemplary port apparatus 2700, that includes the tissue interface component 1010 of FIG. 10, (which can instead be the body 810 in an alternate embodiment, or another arrangement for example). In this exemplary embodiment, the second module 2720 establishes a therapeutic substance reservoir 2727 with a constriction pump (not shown). The therapeutic substance can be contained in the reservoir 2727, and the device is configured to be released via channel 2777 when the pressure in the reservoir is increased sufficiently to force the substance out of the reservoir into the channel 2777. In an exemplary embodiment, the reservoir 2727 can be refilled via a syringe by extending the termination into the device 2720 at the proximal end so that it reaches a receptacle to receive and coupled with the end of the syringe. In an exemplary embodiment, a refilling can be achieved via the snake arrangement detailed above. In some embodiments, the reservoir can be removed from the device 2720, and in other embodiments, the entire device 2720 is removed from the tube 1010 while the tube 1010 remains attached to the tissue of the cochlea.

In an exemplary embodiment, the pumping action can be achieved by constricting the reservoir 2727. In an exemplary embodiment, device 2720 is configured to apply a controllable pressure to the reservoir 2727, so that the therapeutic substance therein travels through a one way port in the reservoir that is in fluid communication with the channel 2777. A sufficiently high pressure in the reservoir overcomes a check valve to open such and permit the substance to flow into the channel 2777.

Positive pressure inside the therapeutic substance containing reservoir 2727 can be used as driving force to release the therapeutic substance in a sustained manner. Positive pressure inside the reservoir can be achieved as noted above, in other ways. By way of example, at least a portion of the boundary wall of the reservoir is made from elastic material which expands when filled with a therapeutic substance containing solution or gel. The expanded elastic material maintains a positive pressure inside the reservoir and when the valve is opened, this existing pressure causes the therapeutic substance to leave the reservoir (thus reducing the pressure a certain amount). Providing that pressure inside the reservoir is higher than the pressure inside the cochlea, therapeutic substance will continue to leave the reservoir. This embodiment does not require a device to apply pressure to the reservoir. Instead, by simply opening and closing the valve, the existing pressure in the reservoir can be utilized to move the therapeutic substance.

In an exemplary embodiment, a gassing agent is filled into the reservoir, such as, for example, a substance that contacts water in the reservoir and causes the gassing agent to change from solid or fluid state into the gas state which increases the volume uptake of the agent and therefore the internal pressure. And in some embodiments, an electrically driven phase change from liquid to gas through electrolysis of water for example using electrodes inside the reservoir or in a separate reservoir can be used to increase the pressure.

The above described positive pressure can be directly applied to the drug inside the reservoir, or indirectly through compressing the drug containing reservoir by pressure build up inside a separate compartment which is in direct contact with the drug containing compartment. In some embodiments, the drug release rate is controlled by the pressure difference between inside and outside the drug reservoir and can be further regulated through valves or through the size/flow resistance of outlet holes/pores. The size of such pores can change with pressure using, for example, perforated stretchable membranes, etc. Such semipermeable membranes can also prevent passing of pathogens or otherwise reduce the passing of such.

Embodiments can enable the concept of treatment of the inner ear where only one portion of the inner ear and/or the barrier that establishes a barrier between the inner ear and the middle ear, is put “at risk” at one time. Accordingly, if a problem arises, and the implant and/or a device cannot be utilized, a workaround can be implemented at another, “virgin” location.

In an exemplary embodiment, the port device detailed herein can enable audiological testing. In an exemplary embodiment, the port device can include a device that is configured to enable testing of one or more aspects of the inner ear. By way of example only and not by way of limitation, the port device can include a module that outputs an electrical signal, albeit for a limited amount of time. The signal could be strong enough and temporally long enough for the recipient to react to such in a conscious manner. That said, NRT and/or ECAPs technologies can be utilized, and in some embodiments, the module of the port device includes one or more components to enable such. Mechanical stimulation can be provided, such as, for example, a module that induces waves of fluid motion for a limited time within the cochlea. In this regard, the component that provides the stimulation is not a hearing prosthesis per se, but can enable a perception of sound for the limited purposes of testing. In this regard, in an exemplary embodiment, if there is no reaction to the mechanical stimulation of the fluid in the cochlea, but there is a reaction to the electrical stimulation, it can be deduced that the cilia have experienced a deleterious scenario, or are experiencing a deleterious scenario (ranges can be tested—say there is only a limited perception with the mechanical stimulation relative to the electrical stimulation). In this regard, upon such a determination a therapeutic substance can be delivered, such as one that preserves the cilia or otherwise might reverse the deterioration of the cilia. And all of this can be combined in one single module or a plurality of modules that communicate with one another and/or communicate with an external device. By way of example only and not by way of limitation, the testing could be activated once a day or could be activated upon an external control signal provided to the implant, and then feedback can be given by the recipient (or automatically in the case of NRT or ECAPS or any of the other applicable objective testing regimes that can have utilitarian value with which the art can enable the utilization thereof with the port's detailed herein), and then the inner ear prostheses or another inner ear prostheses can engage in a therapeutic substance delivery regime or some other regime. Alternatively, this can simply be an indicator that a subsequent procedure can be utilitarian (e.g., the recipient should be provided with a cochlear implant electrode array).

In view of the above, it can be seen that in an exemplary embodiment, the sensors can enable the execution of electrophysiological measurements including, for example, electrical impedance spectroscopy, NRT, EcochG and/or CAP. In an exemplary embodiment, the sensors are configured to assess proteins, pharmaceuticals and/or other molecules in inner ear fluid, new cells and/or bone formation. In an exemplary embodiment, the sensor(s) that are utilized with the port herein can detect and/or measure ECOHG. In an exemplary embodiment, the detection and/or measurement is coupled with a delivery of a test signal to the recipient, and embodiments include devices that enable such. In an exemplary embodiment, the implant is configured to read or otherwise receive the results and change a therapeutic substance delivery regime based on the results. In an alternate embodiment, this information can be provided extra the recipient, and control signals can be provided to the implant from external the recipient in accordance with the teachings detailed herein.

In some exemplary embodiments, the sensors of the port device can be sensors for pH measurements, temperature, pressure, gait, acceleration and/or movement, and as will be understood, some of the sensors may not necessarily directly interface with the perilymph.

Exemplary embodiments include the utilization of the data obtained by the sensors to assess the health of the human in which the port device is implanted, to control the drug release into the inner ear as detailed above, and/or to assess the efficacy of a therapeutic substance being delivered by the implanted device.

Embodiments that utilize impedance based biosensors can provide data on the inflammatory state of the cochlea by for example measuring the concentration of specific proteins which serve as inflammation biomarkers in perilymph. This data can be used to
Operate a therapeutic substance administration system such as a pump or any of the other devices detailed herein to increase or lower the therapeutic substance concentration in perilymph or blood. This can be done in a closed-feedback loop by way of example.
Inform the doctor or patient or caregiver to administer a therapeutic substance orally or intravenously or both.
Inform the doctor or other person whether a therapeutic substance is effective, such as, for example, reducing inflammation. This can widely entail therapeutic substance efficacy monitoring.
To warn a recipient which can be particularly helpful in a patient with episodes of vertigo such as Meniere's patients.

Embodiments that utilize sensors to conduct electrophysiological measurements including by way of example only and not by way of limitation, electrical impedance spectroscopy, NRT, EcochG, and CAP can be used to:
Assess the hearing state of the human over time;
Assess if and how affective a therapeutic substance therapy is to protect or regenerate hearing over time.

Embodiments that utilize impedance based sensors can measure the concentration of specific molecules including drugs in perilymph to
Confirm successful delivery;
Adjust local or systemic dosage to reach target concentration in perilymph.

Sensors such as pH, temperature, pressure, gait, acceleration and/or movement sensors like gait sensors and/or temperature sensors can measure the overall health of a human to
Inform whether a therapy is effective
Warn the patient which can be particularly helpful in patients with episodes of vertigo such as Meniere's patients.

Portions of the sensor(s) can be placed on an antenna like elongated structure that extends from a port in the base of the cochlea deeper into scala tympani towards the apex of the cochlea can be used in some embodiments. By way of example, a “boom” extending from port device 810 can extend less than, more than or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 mm or more or any value or range of values therebetween in 0.1 mm increments from the base of the cochlea. The boom can support, for example, electrodes or other sensors. This can enable sensing at tissue at apical locations of the cochlea. In an exemplary embodiment, the boom can be flexible so as to accommodate the curvature inside the cochlea ducts.

In view of the above, it can be seen that in an exemplary embodiment of the implanted device, the device can include an active component of the device that is a sensor, and the system can include a closed-loop feedback sub-system to control itself to deliver specific amounts and/or types of therapeutic substance.

In an exemplary embodiment, the teachings detailed herein are utilized as part of a method to, and/or the teachings detailed herein are utilized with a device and/or system configured to, treat Meniere's Disease and/or another chronic disease and/or to treat age-related hearing loss. In an exemplary embodiment, the teachings detailed herein are utilized as part of a method to, and/or the teachings herein are utilized with a device and/or system configured to, treat tinnitus, such as by way of example, suppress the perception of tinnitus. In an exemplary embodiment, the teachings detailed herein are utilized as part of a method to, and/or the teachings herein are utilized with a device and/or system configured to, treat an autoimmune scenario with respect to the inner ear, or some other inner ear disease, or a disease that affects otherwise has a deleterious effect on the function of the inner ear. By way of example only and not by way of limitation, embodiments can include enabling the provision of a steroid being supplied to the inner ear all the time.

By way of example only and not by way of limitation, in an exemplary embodiment, the teachings detailed herein are utilized as part of a method to treat balance and/or vertigo. In an exemplary embodiment, the teachings detailed herein are executed to enable a human who previously was not able to drive a vehicle or otherwise operate machinery in a safe manner, including in a scenario where a licensed organization or a supervisory organization (e.g., a Department of Motor Vehicles) previously deemed the person unable to do so. Accordingly, exemplary methods include receiving permission from such organizations to again continue executing one or more of these actions.

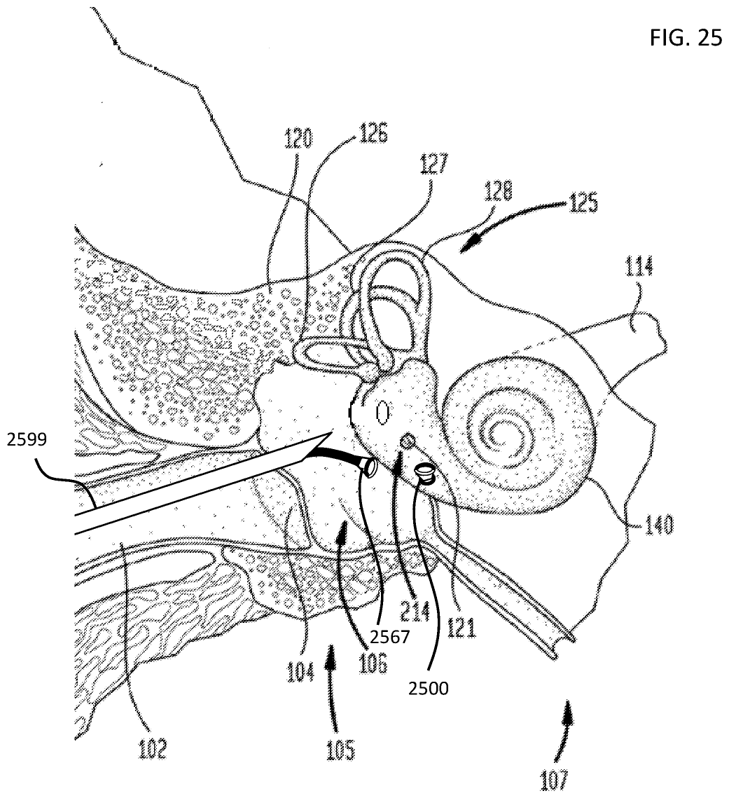

In an exemplary embodiment, the inner ear port device includes a magnetic alignment feature that can enable the action of blindly finding the inner ear port device utilizing a trans tympanic membrane approach. By way of example only and not by way of limitation, FIG. 25 presents an exemplary scenario where, for example, termination 2599 extends through the ear canal 102 and through the tympanic membrane 104, such that the entire tip and a portion of the shank of the termination is located in the inner ear cavity 106. The termination is part of an insertion tool that enables or otherwise includes “snake” 2567. Snake 2567 is a guidewire like device that is flexible and is hollow and configured to provide for transportation of the therapeutic substance from the tool or from a location remote from the tool, through the snake 2567, and out the exit of the stake 2567. At the end of snake 2567 is a nozzle that includes two “C” shaped magnets (the Cs are exactly half circles). The polarity of these magnets is opposite one another. As shown, the darker colored magnet is such that the north pole faces towards the rest of the snake and the light colored magnet is such that the north pole faces away from the rest of the snake.

Inner ear port 2500 is shown located in the lower portion of the cochlea below and to the right of the round window 121. This feature is different than the arrangement shown in FIG. 7, where the port 700 accesses the side of the cochlea where the oval window is located. As seen, inner ear port device 2500 includes a magnet arrangement that corresponds to that of the snake 2567. Accordingly, when the snake 2567, or more specifically, the end of the snake 2567 reaches a location proximate the port 2500, the magnetic attraction between the two components will cause the snake to the moved towards an otherwise guided on to the port 2500 in an aligned manner, owing to the polarity of the magnets in the arrangements of the magnets on each of the components. Upon sufficient coupling, the therapeutic substance can be transferred from the snake to the port 2500, and into a reservoir, for example, to refill the reservoir. Accordingly, by utilizing the magnets, in an exemplary embodiment, the snake 2567 can be “blindly” inserted into the middle ear, and potentially at least towards a rough idea of where the port is, and then the magnetic attractions takeover to guide the snake to the port. Accordingly, exemplary embodiments can include accessing the port and transferring a therapeutic substance to the port device without being able to visually see the port, whether directly or by utilizing a camera.

In an exemplary embodiment, there can be a magnetically actuated valve that is located in the port device 2500, that opens when the snake 2567, or more specifically, when the magnets of the snake 2567 become located proximate the port, thus to enable the transfer of the therapeutic substance from the snake to a reservoir. The magnetically actuated valve can be such that when the snake is pulled away from the port 2500, such as by way of example, when the snake 2567 is pulled back through the termination 2599, the absence of the magnetic field will cause the valve to close. It is noted that the magnetic field could cause this opening, and thus can provide the “force” to open and close the valve, while in other embodiments, the port device 2500 can include a sensor or otherwise an electronic logic circuit that, when the magnetic field is sensed, the port device 2500 controls itself to open. That said, instead of utilizing magnetic fields per se, a signal could be provided from the snake to the implant 2500 instructing the prostheses 2500 to open the valve. Such an arrangement can be utilized with embodiments that do not necessarily utilize the snake 2567. For example, if a termination is utilized to directly access the port device, a communication signal can be provided from external the recipient and/or from internal the recipient (an antenna can be located on the termination), to the port device, to instruct the port device to open the valve and/or close the valve.

Corollary to this is that in some exemplary embodiments, the valve of the port can be spring loaded or otherwise biased close, and then when a male portion of the snake and/or the termination enters the passage, the male portion pushes the valve open, and then upon withdrawal, the valve “springs” shut, thus preventing the therapeutic substance for example, from entering the middle ear or otherwise escaping from the cochlea, or otherwise limiting the amount of perilymph escape relative to that which would otherwise be the case.

And in a further embodiment, there can be a mechanical actuation system that opens the valve and closes the valve. By way of example only and not by way of limitation, there could be a receptacle for a screw driver or a hex driver or the like on the portion of the port that is located in the middle ear, and by turning this receptacle, because a mechanical linkage between the receptacle and the valve, the valve can be turned open and turned closed.

It is also noted that the embodiment of FIG. 25 can be utilized to couple a data transfer connection to a connector of the port device. As noted above, the port device can include a memory chip or the like. Here, the apparatus of FIG. 25 can guide a connector transported by the termination 2599 to the port device 2500, and the above noted magnets can be utilized to place the connector into contact and couple with the connector of the port device, upon which data transfer can be executed. In an exemplary embodiment, an RF inductance coil can be located with element 2567, and the port device can also include an inductance coil, and the magnets can be utilized to align the inductance communication purposes.

Any device or system that enables and/or any method of guiding a therapeutic substance delivery device to the port device and/or any device or system that enables and/or method of opening and/or closing a valve so the reservoir can be refilled can be utilized in at least some exemplary embodiments providing such is enabled by the art unless otherwise noted.

And further, while the above embodiment has been described in terms of transferring therapeutic substance that is in the form of a fluid, in other embodiments, the therapeutic substance can be in a solid form or otherwise contained in a solid container. In this regard, solid pellets or solid containers can be pushed through the snake, by a guidewire for example, and then into the port device.

In view of the above, it can be seen that in some exemplary embodiments, such as where the grommet is utilized to establish the tissue interface component, or body 810 in an alternative embodiment, or any other arrangement where a body or grommet is firmly screwed into the bony tissue through which the cochleostomy is made (where the action of screwing could establish the cochleostomy in the case of a self-tapping grommet or self-tapping body 810), which body or grommet establishes a passageway from the middle ear to the inner ear, this passageway can be utilized as an access hole for one or more purposes, such as by way of example only and not by way of limitation, the utilization of the delivery of the desired or needed drugs steroids fluids and/or tissue growth inhibiting substances, all by way of example, to the inside of the cochlea. And again, in at least some exemplary embodiments, there are methods where, when access is not needed, the access hole is plugged or otherwise sealed to prevent fluid within the cochlea from escaping into the middle ear.

In an exemplary embodiment, the implanted dedicated port is part of an implant that is configured to regulate release of the transferred substance into the inner ear, and the method includes regulating the release of the transferred substance over a period of at least 0.25, 0.5, 0.75, 1, 1.25, 1.5, 1.75, 2, 2.5, 3, 3.5, 4, 5, 6, 7, 8, 9, 10, 11, 12, 13, 14, 15, 16, 17, 18, 19, 20, 21, 22, 23, or 24 hours or 1, 2, 3, 4, 5, 6, 7, 8, 9, 10 days or 1, 2, 3, 4, 5, 6, 7, 8, 9, 10, 11, 12, 13, 14, 15 or more weeks or months, or any value or range of values therebetween in 0.1 hour increments, using the port.

In an exemplary embodiment, the action of obtaining access is executed at least 1, 2, 3, 4, 5, 6, 7, 8, 9, 10, 11, 12, 13, 14, 15, 20, 25, 30, 35, 40, 45, 50, 60, 70, 80, 90 or 100 days or weeks or months or any value or range of values therebetween in one day increments after the implanted dedicated port was fully implanted in the human.

Embodiments further include transcutaneously enabling the second function, such as transcutaneously enabling the transfer of substance via a wireless transcutaneous communication system. In an exemplary embodiment, as detailed above, the port includes a wireless receiver that receives a wireless signal transmitted as part of the action of enabling. In an exemplary embodiment, the action of transcutaneously enabling is executed at any one or more of the temporal periods detailed herein (e.g., 60 days after the implanted dedicated port was fully implanted in the recipient). In an exemplary embodiment, the action of transferring can be executed for at least 1, 2, 3, 4, 5, 6, 7, 8, 9, 10, 11, 12, 13, 14, 15, 20, 25, 30, 35, 40, 45, 50, 60, 70, 80, 90, or 100 minutes, or hours, or any value or range of values therebetween in 0.1 minute increments.

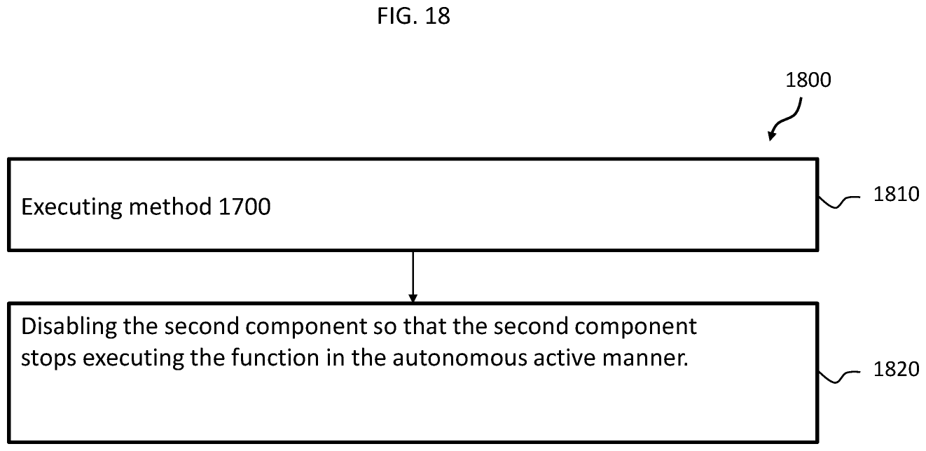

FIG. 18 presents an exemplary flowchart for an exemplary method, method teaching 1800, according to an exemplary embodiment. Method 1800 includes method action 1810, which includes the action of executing method 1700. Method 1800 further includes method action 1820, which includes the action of disabling the second component so that the second component stops executing that function in the autonomous active manner. This could entail placing the second component into a standby mode for example. The device could regain or otherwise re-implement the autonomous actions when instructed (enabled), but for the time being, it does not execute the function. In an exemplary embodiment, this can include transcutaneously disabling the second component via a wireless transcutaneous communication system. In an alternate embodiment, this can be executed by providing a magnetic field to a switch that responds to a magnetic field to shutdown or otherwise disable the second component. In an exemplary embodiment, this can be executed by inserting a guidewire or the like through the tympanic membrane to the second component and placing the guidewire in contact with a sensor or the like. A magnet could be located at the end of the guidewire, where the magnet will activate the magnetic switch. Alternatively, the magnet could simply aid in the guidance of the guidewire to the second component, and upon contact with the second component, the enable/disable switch of the second component could be moved to the disable location.

In an exemplary embodiment, any one or more of the actions detailed herein with respect to enabling, disabling and/or transferring can be executed at least 1, 2, 3, 4, 5, 6, 7, 8, 9, 10, 11, 12, 13, 14, 15, 20, 25, 30, 35, 40, or 50 times or more, or any value or range of values therebetween in one increment while the same port, or at least the portion of the port that interfaces with the tissue establishing the passageway, is the same.

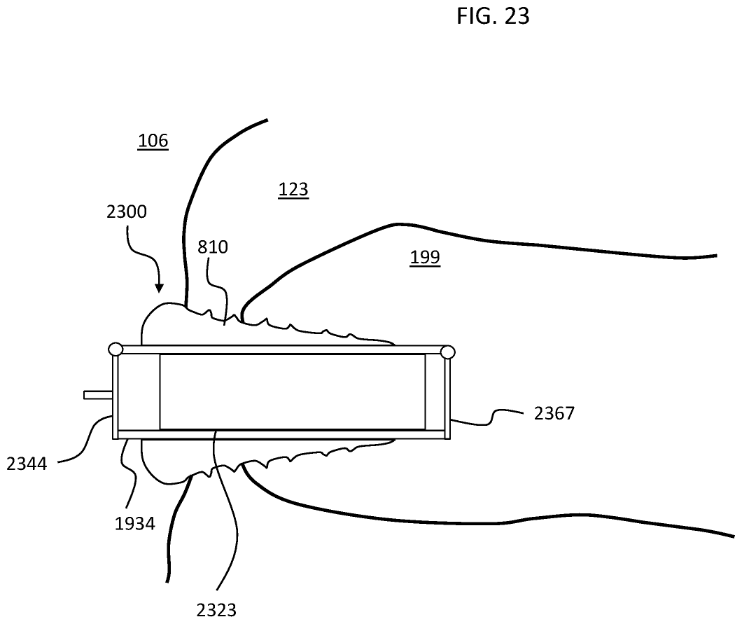

FIG. 23 presents another exemplary embodiment of an inner ear device 2300. Here, there is the tissue interface body 810, which can correspond to the body detailed above. Here, the second module is established by a tube 1934, and there is a hinged spring loaded door 2344 with a nub facing outward to enable a tweezers or the like to grip such and provide a force to counteract the spring load to open the door. This is an exemplary embodiment where the artificial round window or oval window is located at the proximal end of the prostheses, an embodiment briefly mentioned above. This exemplary embodiment of implant 2300 further includes a therapeutic substance delivery submodule 2323 of the second module. In this exemplary embodiment, element 2323 can be a reservoir that contains a therapeutic substance, where the therapeutic substance is configured to defuse through the outer wall thereof. In an exemplary embodiment, element 2323 can be a body of the therapeutic substance in a solid configuration, and the solid configuration will dissolve or elute when exposed to the perilymph. This is controlled by controllably opening and closing door 2367, where door 2367 is connected to an actuator under the control of the implant and/or controlled by an external device (active control of this is disclosed below). The door can be opened for a period of time to allow perilymph to flow into the second module, and can remain open for a period of time, while the therapeutic substance dissolves or a substance elutes into the perilymph in the second module. Alternatively, the door could open for a shorter period of time to allow perilymph into the second module, and then the door could shut, and then the perilymph could dissolve partially the therapeutic substance 2323 elution could take place, etc., and then upon a predetermined time and/or after a sensor determines the concentration of the therapeutic substance within the perilymph in the second module, door 2367 could open allowing the only thing in the perilymph combined with the therapeutic substance to mix with the perilymph in the duct(s) of cochlea 199. Active control of the door 2367 can control the rate of dissolution/elution. This could repeatedly occur in a manner that is controlled to thereby control the concentration of the therapeutic substance within the cochlea. Still, in an exemplary embodiment, door 2367 could remain open for a period of time sufficient to achieve the desired concentration of therapeutic substance into the cochlea, and then door 2367 could close again, and then this could be repeated.

In an exemplary embodiment, a blower device, such as an impeller can be utilized to circulate perilymph past the therapeutic substance within the second module.

In an exemplary embodiment, one of the modules attached body 810 can be a device that repeatedly “shoots” solid balls (tiny solid balls) of therapeutic substance into the cochlea, where the balls dissolve in the perilymph. In an exemplary embodiment, one of the modules attached to the body 810 can be a device that gradually and/or periodically feeds a solid stock of therapeutic substance into the perilymph, where the solid stock can dissolve as it is driven or otherwise moved into the cochlea or otherwise to interface with the perilymph therein. This could be analogous to a welding stick for example, or more accurately, a welding wire that is fed out a nozzle at a controlled rate during welding. In an exemplary embodiment, this can have utilitarian value with respect to maintaining a “clear” cochleostomy/port area. A constant and/or periodic feed can inhibit sealing off of the area by fibrotic tissue by way of example. And indeed, in some embodiments, such a technique can be utilized irrespective of the utilization of a therapeutic substance. By way of example only and not by way limitation, the port can include a device that periodically or continuously “clears” the area around the port or otherwise the area of importance related to the port, such as the area around the opening/passage through the port. In an exemplary embodiment, something along the lines of a rigid windshield wiper for example might be utilized, albeit constructed and arranged otherwise configured for utilization in the ear system of a human. A retractable ram can be utilized by way of example. Any device system and/or method that will actively clear the area of tissue that would grow or otherwise could grow over time to maintain the utilitarian value of the implant can be utilized in at least some exemplary embodiments.

Still further, embodiments can include the delivery of solid bodies that contain a therapeutic substance in a fluid state, where the solid bodies can dissolve to thus release the deputy substance therein. Alternatively, and/or in addition to this, the solid bodies can split otherwise open to release the therapeutic substance when in the interior of the cochlea.

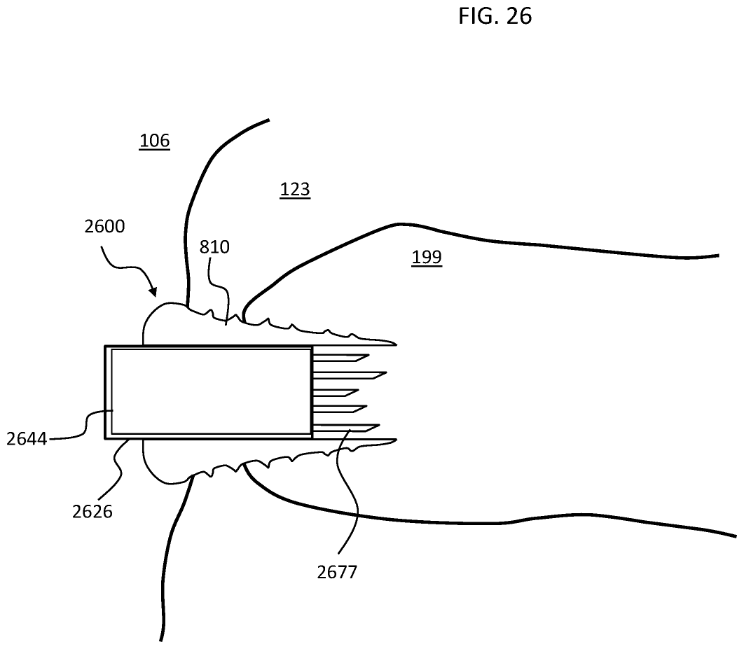

FIG. 26 provides another exemplary embodiment of an inner ear device 2600, that includes the body 810 detailed above, into which is removably threaded a second module, which includes a housing 2626, in which is located a reservoir 2644 containing a therapeutic substance. The reservoir 2644 is in fluid communication with a plurality of delivery ports 2677. In an exemplary embodiment, a porous membrane can be located between the therapeutic substance contained in the reservoir and the delivery ports, to meter or otherwise slow the delivery of the therapeutic substance. In an exemplary embodiment, the reservoir can be controllably pressurized to control the delivery of therapeutic substance from the one or more delivery ports 2677. In an exemplary embodiment, the housing 2626 can be unscrewed from the body 810, and then a new housing can be replaced with additional therapeutic substance or new types of therapeutic substance. Further, in an exemplary embodiment, the housing can be opened and the reservoir 2626 could be swapped out with a new reservoir. In an exemplary embodiment, the aforementioned membrane can be such that the membrane permits transfer of fluid in only one direction (i.e., into the cochlea), so that when the reservoir is removed for replacement, but perilymph does not leak or otherwise escape out of the cochlea, or otherwise limits the amount of perilymph that could leak out of the cochlea relative to that which would otherwise be the case.

Embodiments can also include a method of accessing the cochlea utilizing a traditional cochleostomy and/or by entering the cochlea utilizing the round window or utilizing the oval window, which cochleostomy or entrance by the round window or entrance by the oval window is utilized to insert a cochlear implant electrode array into the cochlea. Then, during the same procedure of accessing the cochlea as just noted, a second cochleostomy or a first cochleostomy is established for the inner ear port device. This could enable future access to the cochlea without having to perform another entire surgery to access the cochlea. That is, by way of example only and not by way of limitation, this can be analogous to changing a timing belt when accessing other portions of an engine for maintenance or repair, even though the timing belt does not need to be changed per se. That is, the difficulty in accessing the location far outweighs the de minimis nature of taking an action that never has any future utility in practice and and/or utilizing a device that will never be used in the future or ever at all. Accordingly, exemplary embodiments include inserting a cochlear implant electrode array through a first passage, and inserting an inner ear port device into a second passage from the middle ear into the inner ear, and not utilizing that inner ear port device for one or more or all of its intended purposes for at least 1, 2, 3, 4, 5, 6, 7, 8, 9 or 10 years or more or any value or range of values therebetween in one month increments from the time of implantation. In an exemplary embodiment, the inner ear port device is utilized at the time of implantation, but that is not utilized again for any one or more of the aforementioned temporal ranges.

In an exemplary embodiment, there is the aforementioned tissue interface body that provides a passage from the middle ear to the inner ear, where the passage is plugged for any one or more of the aforementioned temporal periods. That is, in an exemplary embodiment, the access hole is plugged when not in use. In an exemplary embodiment, the access hole is plugged in a manner that prevents any impact on the auditory system.

In an exemplary embodiment, the port device is completely unrelated to any function in the auditory system. In an exemplary embodiment, the port device is related to a function of the auditory system, such as where the port device is utilized with the cochlear implant electrode array as noted above.

An exemplary method includes utilizing a body that comprises a biosuitable material to establish a permanent tissue interfacing implant that provides a passageway from the middle ear into the inner ear. In an exemplary embodiment, the aforementioned biosuitable material causes a mammalian inflammatory response, and this can be utilitarian with respect to providing a seal between the tissue (wall of the cochlea through which the body passes) and the body. In any event, at least some exemplary embodiments of the tissue interfacing body integrate in a utilitarian manner with the cochlear bony wall. A second component or second module is placed or otherwise is located in the passageway so as to fluidically seal the cochlea with respect to the passage that has been created, in which the implant is located.

It is noted that at least some exemplary embodiments include providing a “universal” tissue interface body that establishes a passage between the middle ear and inner ear. By way of example only and not by way of limitation, this can correspond to the body 810 detailed above by itself. The body can include a threaded passage therethrough, into which the threaded passage can initially be threaded a cap that will seal the passage and prevent fluid leakage from the inner ear to the middle ear. This cap can be considered a second module, and can be replaced with, in the future, another module that has one or more of the features and/or structural components detailed herein, or any other functional or structural component that can have utilitarian value. This can enable the functionalities to be changed in accordance with temporally changing needs of a recipient. Alternatively, and/or in addition to this, embodiments include a kit arrangement where, for example, the kit includes a tissue interface component, such as body 810, and then a number of different second modules that have various functionalities. In an exemplary embodiment, this can enable a surgeon or otherwise a healthcare professional to basically “build” an implant according to the needs at the time of assessment. By way of example, a kit could permit the establishment of a therapeutic substance delivery device or a sensor device etc., where the surgeon could “screw” a second component (or a first component, for that matter) into the threaded passage of the body 810, that corresponds to one or more or all of the components that are attached to the body 810 detailed herein. This can be done before or after implantation of the body 810. By way of example only and not by way of limitation, the surgeon could “build” a combined therapeutic substance delivery system and a cochlear implant system. The surgeon could build a sensor implant and/or a sensor combined with a drug delivery implant. Any one or more of the combinations detailed herein can be combined with any one or other of the combinations detailed herein providing that the art enables such unless otherwise indicated. Accordingly, embodiments include methods of establishing any one or more of the combinations any one or more of the features detailed herein, and these methods can be executed by a healthcare professional such as the surgeon or someone under the supervision of the surgeon or otherwise working with the surgeon, and this can be done within 1, 2, 3, 4, 5, 6, 7, 8, 9, 10, 11, 12 hours or 1, 2, 3, 4, 5, 6, 7, 8, 9, 10 days of the implantation of the device, and can be done within half a mile or within a mile of the implantation site.

It is also noted that some exemplary embodiments include the ability to “lock” the second module, whatever it is, to the first module. In an exemplary embodiment, upon locking, the second module cannot be removed from the first module without removing the first module from the tissue/bone.

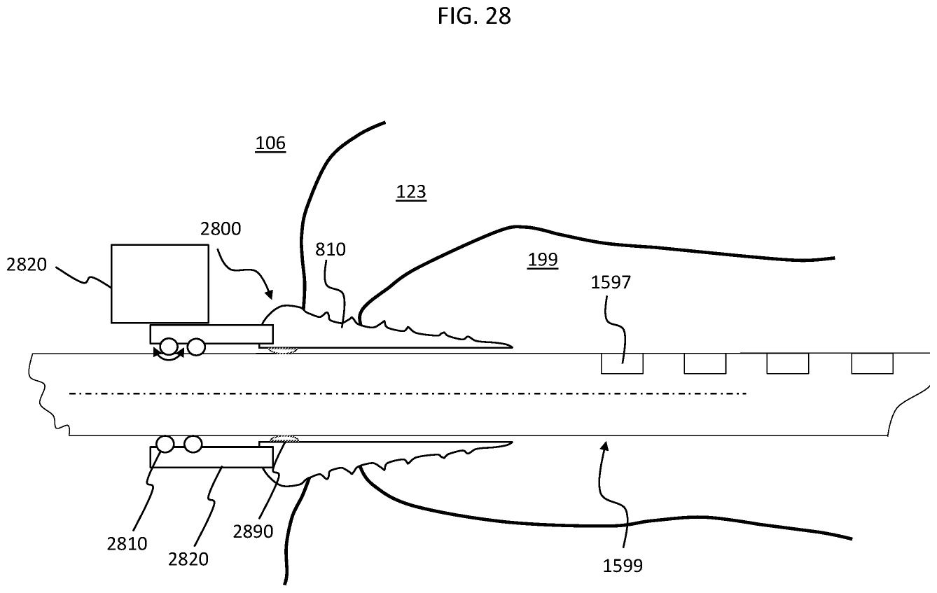

FIG. 28 presents a port device that has an electrically driven mechanism for automated advancement of a cochlear implant electrode array 1599 into the cochlea duct 199. In the embodiment shown, advancement speed and/or direction of the electrode array is controlled through 1 or more electrically driven rolls 2810 which are in contact with the electrode array and move the array through friction.

More specifically, the port device 2800 shown includes the body 810 as described above or a variation thereof. An interior seal 2890 can be attached to the body 810, and permits the electrode array 1599 to slidingly move therethrough, but also provide a seal so as to prevent perilymph from escaping. Here, a second module that includes tube 2890 is screwably attached to the body 810. Tube 2890 supports electric motor 2820, which is in mechanical communication with the rollers 2810. By any of the control regimes detailed herein, actuation of the rollers move the electrode array 1599 inward and/or outward. Speed and direction can be controlled by a person or through a closed feedback loop using electrophysiological measures, such as impedance, EcochG, NRT and/or pressure sensing data. In an exemplary embodiment, a recipient who initially suffers from only high frequency hearing loss as the electrode array inserted into the cochlea only such that perhaps the first three or four or five or six or seven or eight or nine or ten electrodes (out of a 22 electrode array (electrodes evenly spaced in some embodiments)) or any value or range of values therebetween in 1 increment are located in the cochlea or otherwise able to stimulate tissue of the cochlea owing to their position. Then, as time progresses, the recipient loses hearing in the mid-and/or lower frequencies. In an exemplary embodiment, the device 2800 is controlled so that the rollers 2810 drive the electrode array 1599 further into the cochlea so that additional electrodes are located in the cochlea or otherwise are able to stimulate tissue owing to their position within the cochlea. In an exemplary embodiment, upon the movement of the electrode array into the cochlea, an additional 1, 2, 3, 4, 5, 6, 7, 8, 9, 10, 11, 12, 13, 14, 15, or 16 electrodes or more, or any value or range of values therebetween are located within the cochlea. Control can be executed by any of the wireless regimes detailed herein. Alternatively, and/or in addition to this, a portion of the implant 2800 could be physically accessed, and the electric motor 2820 could be activated accordingly. In an exemplary embodiment, a voltage could be applied to the electric motor 2820 to actuate the motor in one direction (and an opposite voltage could be applied to reverse the electric motor).

As can be seen, there can be utility with respect to enabling a treatment of a person afflicted with loss of hearing such that only one major operation need be executed: the original implantation of the port device and cochlear implant. By partially implanting the entire array into the cochlea, residual hearing in the medium and lower frequencies can be preserved. When (if) that hearing degrades, the electrode can be further advanced into the cochlea without having to undergo any surgery at all, or at least without having to undergo major surgery.

Moreover, in an exemplary embodiment, the implantation of the electrode array could be a precaution that is executed during a procedure to access the cochlea for other purposes. In this regard, in an exemplary embodiment, consistent with the teachings detailed herein, the port devices herein have utilitarian value with respect to utilization beyond that associated with the stimulating device, such as a cochlear implant. As detailed above, the port devices have utilitarian value with respect to providing therapeutic substance and/or providing a sensor device. In these devices would be utilized to treat or otherwise mitigate hearing loss. However, in some scenarios, there is potentially an intractable possibility, including a statistical likelihood, that hearing will be lost, and thus a cochlear implant may be needed. Accordingly, the cochlear implant electrode array could be positioned for future insertion into the cochlea or otherwise partially inserted into the cochlea, but positioned in a manner that does not affect the hearing that is present in the recipient. During this time, the teachings detailed herein can be utilized to treat the cochlea, such as providing a therapeutic substance to the cochlea utilizing the port device and/or sensing phenomenon signed the cochlea. But at some point, the hearing may fail, and thus the “pre-positioned” cochlear implant electrode array can be inserted into the cochlea and electric hearing can be commenced thereafter.

Thus, in an exemplary scenario of use, the port devices are used for the sole purpose of delivering drugs to the inner ear of patient/recipient for a first period of time and then the port device facilitates the insertion of a cochlear implant electrode array at a later time point in time. The use of the cochlear implant electrode array can be utilized simultaneously with further therapeutic substance delivery and/or sensors associated with the port device. Thus, in an exemplary embodiment, there is a port device that has dual (at least dual) or triple (at least triple) functionality: any two or three of the combination of a sensor device, a therapeutic substance delivery device and a cochlear implant. In an exemplary embodiment, the utilizations of the functionalities are staggered, and in some embodiments, the utilizations can overlap. In an exemplary embodiment, any one or two of the functionalities are utilized for at least 1, 2, 3, 4, 5, 6, 7, 8, 9, 10, 11, 12 days or weeks or months or years or any value or range of values therebetween in one increments of the associated unit, and then anyone all other or two other of the functionalities are then implemented and utilized (the initial one or two functionalities are not utilized prior, aside from, potentially, testing and validation that such will be utilitarian when later implemented).

It is noted that in some embodiments, the implantable component(s) can include some active components or otherwise some smart components, such as, for example, components that can control the valves detailed herein. By way of example, the implantable component can include a long life power source, such as a long life battery (relative to the application) and a valve controller (circuitry, such as a logic circuit configured to control the valve, which can include a clock circuit, etc., a processor, a chip, a miniature computer device programmed to control the valve, etc.) that is powered by the long life battery, wherein the valve can be opened and shut via a micro actuator that is also powered by the long life battery. In an exemplary embodiment, the external component can be configured so as to provide a wireless signal, such as inductance RF signal, to the valve controller (or, an antenna thereof), which can activate the valve controller to open and close the valve. In an exemplary embodiment, the valve controller can be a micro circuit or any other appropriate set of electronics which can receive a signal, evaluate or otherwise determine what the signal means (or simply that the signal is present), and output a command to the valve. That said, in an alternate embodiment, a simple gate switch can be located between the long life power source and the actuator that actuates the valve, where the gate switch reacts to, for example, vibrations from the external component and/or from an inductance RF signal generated by the external component, such that the switch opens or closes, and current is permitted to flow from the long life battery to the actuator (or not permitted to flow), thus causing the actuator to actuate and thus open the valve. In this regard, in an exemplary embodiment, the external component includes the aforementioned RF inductance transmitter and is configured to transmit an inductance signal to the implanted component. When the external component is utilized, or, in some embodiments, simply turned on, and placed against the skin of the recipient, the external component transmits this RF inductance field, which is received by the implanted component, and the implanted component open the valve to enable therapeutic substance transport from the reservoir.

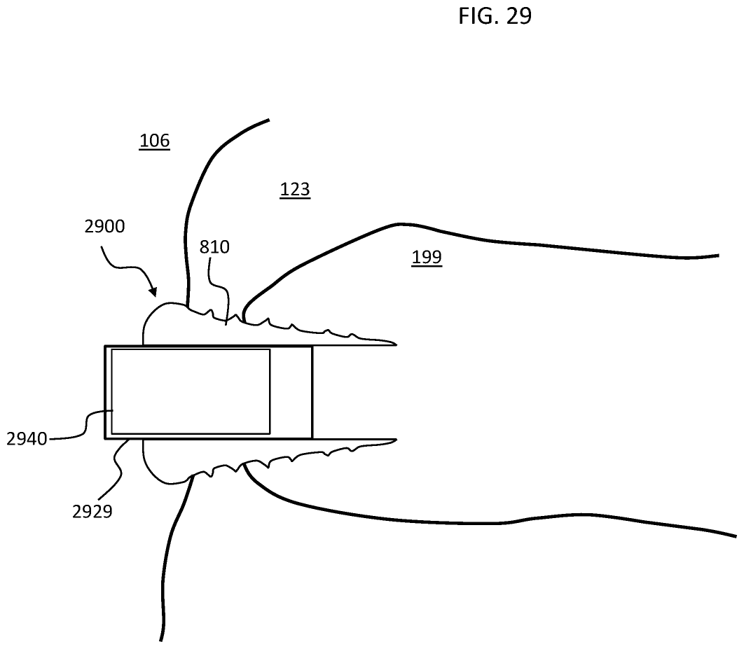

FIG. 29 presents an exemplary inner ear device 2900. This apparatus includes a sensor that is a movement sensor, and the inner ear device is or is part of a balanced device. More specifically, the housing 2929 is located in port 810, thus sealingly closing the passageway therein. Inside housing 2929 is a motion sensor 2940. Thus, by replacing housing 2929, the motion sensors can be replaced. Also, by removing the housing 2929, the duct 199 can be accessed in accordance with the teachings detailed herein. There can be utilitarian value with respect to placing the motion sensor at the inner ear. The closer that the motion sensor is to the center of the human, the more utilitarian the sensor becomes. That is, by measuring movement closer to the center of the human, left/right issues are reduced (e.g., an action of turning would have different results if the measurement was taken on the left side of the head versus the right side of the head, whereas measurements taken at the center of the head would present true data). And it is noted that while the embodiments detailed herein are directed towards port devices that are utilized to access the ducts of the inner ear, the port devices can be utilized to simply support such sensors (the port may not necessarily extend to a cavity-the port can be utilized as a mere bone fixture for example).

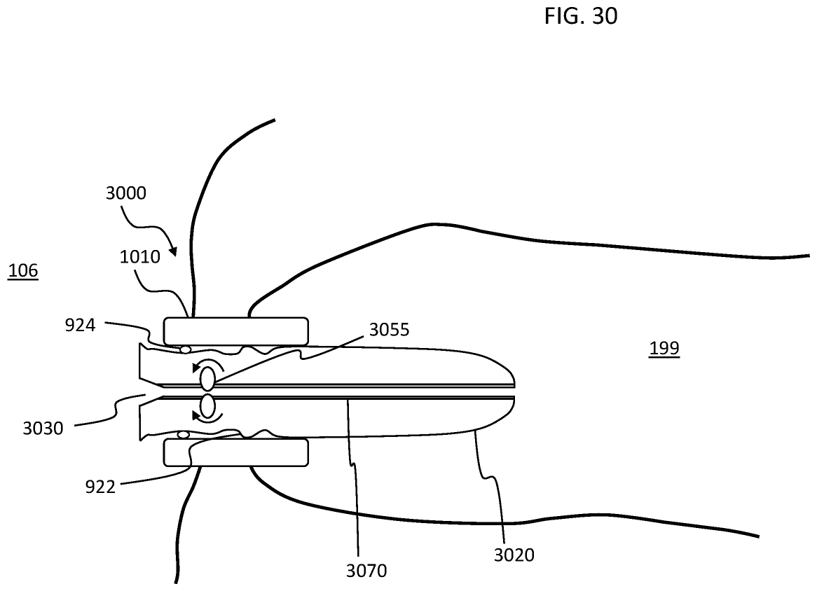

In this regard, FIG. 30 presents an exemplary port apparatus 3000, that includes the tissue interface component 1010 of FIG. 10, (which can instead be the body 810 in an alternate embodiment, or another arrangement for example). In this exemplary embodiment, the second module 3020 establishes a pump/cannula adaptor with a peristaltic pump (see oval shaped components 3055) that variously constricts cannula 3070 which is a flexible polymer tube in some embodiments. Electric motor actuator (not shown) drives the components 3055 in the direction of the arrows so as to peristaltically pump therapeutic substance through the cannula 3070. In an exemplary embodiment, receptacle 3030 is provided so as to allow connection to a cannula of a drug pump that is external to the recipient/human, which connection can be made in the middle ear. Upon connection to the cannula of the external drug pump, therapeutic substances can be pumped into the duct of the cochlea 199 directly.

The pump/cannula adaptor can include a one-way valve to allow fluid flow into the cochlea and restrict fluid flow out of the cochlea. A filter and/or a semipermeable membrane can allow therapeutic substance to be transported into the cochlea, and can bar pathogens so as to control or otherwise mitigate the risk of infection.

In combination with an electrically powered port device 1010, where electrical contacts can be located along the sides of the second module 3020 to place the second module 3020 into electrical communication with the tissue interface portion 1010, so that power and/or control of the pump can be controlled by port portion 1010. The apparatus 3000 can pump therapeutic substance from the cannula (or from a reservoir-a reservoir can be attached to the receptacle 3030, and thus located in the middle ear) from a location outside the inner ear across the adaptor 3020 into the inner ear. The pumping action can be controlled through external communication as detailed above and/or can be executed in an automated manner such as by close feedback loop utilizing data from sensors. In an exemplary embodiment, an inductance coil is located on the tissue interface portion, and power can be inductively transmitted thereto, and owing to the electrical connection between the tissue interface portion 1010 and the second module 3020, the power from the inductance transmission can be provided to the electric motor in the module 3022 power the pump. Alternatively, and/or in addition to this, logic circuitry and control devices can be located with the tissue interface portion 1010, along with potentially a power source. Again, owing to the electrical communication with the tissue interfacing port 1010 and the module 3020, the tissue interface portion 1010 can control the operation of the pump.

The device of FIG. 30 can have utilitarian value with respect to providing an adaptor for the therapeutic substance supply located outside of the human. In an exemplary embodiment, all that is needed is to snake the cannula from the supply to the adaptor 3020, and connect the cannula to the receptacle 3030. The device of FIG. 25 can be utilized to snake the cannula to the port device. The port device can take over from there (providing that the therapeutic substance can reach the adaptor 3020) and pump the therapeutic substance into the cochlea.

It is noted that any reference herein to a therapeutic substance corresponds to a disclosure of an active substance such as an active drug or an active biologic etc., and any disclosure herein to an active substance such as an active drug or the phrase active substance in the generic manner corresponds to a disclosure of an active biologic or a therapeutic substance, etc. Any active pharmaceutical ingredient that can have utilitarian value can be a therapeutic substance. Proteins can be therapeutic substances as well. It is also noted that in an at least some exemplary embodiments, an inactive fluid can be a physiological saline, which can be utilized to convey the therapeutic substance into the cochlea.

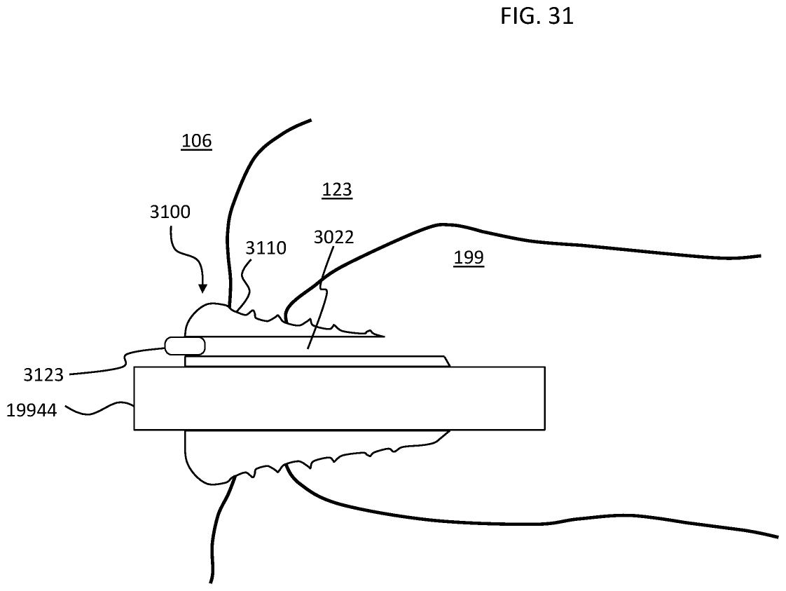

FIG. 31 presents an exemplary implantable port prosthesis 3100 that includes a tissue interface component 3110 that has the traditional passage 822 therethrough as can be seen, along with a second passage 3022. In this exemplary embodiment, a sensor 19944 is permanently fixed in the passage 822 (although in other embodiments, it can be removably fixed). Thus, as can be seen with respect to this embodiment, with a seal 3123 located in the second passage 3022, the inner ear barrier tissue interface includes a second passage that is unsealably sealed, the second passage providing physical access from the middle ear into the inner ear through the second passage bypassing the first functional component (sensor 19944). In this regard, in an exemplary embodiment, a termination can be inserted into passage 3022 from the middle ear side when the plug 3123 is removed and a therapeutic substance can be transported into the duct 199. Alternatively, a borescope or some other device or probe could be inserted through the passage 3022, or some active component for that matter. A second sensor could be located in the second passage 3022.

In an exemplary embodiment, therapeutic substance include but are not limited to, any of those detailed above, and can include peptides, biologics, cells, drugs, neurotrophics, etc. Any substance that can have therapeutic features if introduced to the cochlea can be utilized.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Embodiments include embodiments where any or more of the teachings detailed herein are combined with any one or more of the other teachings detailed herein, unless otherwise noted, providing that the art enables such. Embodiments also include embodiments where any one or more of the teachings detailed herein are specifically excluded from combination with any one or more of the other teachings detailed herein nless otherwise noted providing that the art enables such.

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. It is also noted that embodiments can include devices systems and/or methods that specifically exclude one or more of the disclosures presented herein (i.e., it is not present).

While various embodiments of the present invention have been described above, it should be understood that they have been presented by way of example only, and not limitation. It will be apparent to persons skilled in the relevant art that various changes in form and detail can be made therein without departing from the scope of the invention.

1. A device, comprising:
a body through which a passage extends, wherein
the body is configured to permanently fix to an opening in a barrier between a middle ear and an inner ear of a human,
the device is an inner ear port apparatus that is configured to enable resealable physical access from the middle ear into the inner ear through the passage, and
the inner ear port apparatus includes insulated electrically conductive material configured to conduct an electrical signal.

2. The device of claim 1, wherein:
the body includes electronics which include the insulated electrically conductive material configured to conduct an electrical signal.

3. The device of claim 1, wherein:
the inner ear port apparatus includes electronics which include the insulated electrically conductive material configured to conduct an electrical signal.

4. The device of claim 2, wherein:
the body corresponds to a first module of the device; and
the device includes a second module that is removably attached to the first module, wherein
the second module is in signal communication with the electronics.

5. The device of claim 2, wherein:
the body corresponds to a first module of the device;
the device includes a second module that is removably attached to the first module, wherein
the second module is in electrical communication with the electronics.

6. The device of claim 1, wherein:
the inner ear port apparatus includes a power storage device.

7. The device of claim 1, wherein:
the inner ear port apparatus includes a memory chip.

8. A device, comprising:
a tissue interface portion configured to implantably attach to tissue of and/or proximate an inner ear of a human; and
a powered sensor, wherein
the device is a non-simulative device configured to sense at least one phenomenon related to the inner ear of the human.

9. The device of claim 8, wherein:
the device includes a wireless transcutaneous communication system configured to receive power to power the sensor and/or to transmit data based on data obtained by the sensor.

10. The device of claim 8, wherein:
the powered sensor is part of the tissue interface portion; and
the tissue interface portion has a passage configured to enable access into a cochlea, wherein the passage is unpluggably plugged to seal the passage.

11. The device of claim 8, wherein:
the device includes an onboard power source that provides power to the sensor.

12. The device of claim 8, wherein:
the device enables repeated sealingly access from the middle ear to the inner ear through a closable passage in the device, such that upon closing the passage is fluid tight.

13. The device of claim 8, wherein:
the sensor is a biosensor in contact with perilymph configured to measure a property thereof.

14. A device, comprising:
a tissue interface portion configured for securement to tissue of and/or proximate an inner ear of a human and provide a long term passage from outside the inner ear to inside the inner ear; and
a therapeutic substance container at least indirectly releasably attached to the tissue interface portion and/or a portion of the device supported by the tissue interface portion, wherein
the device is configured to actively control itself and/or to be actively controlled remotely to deliver therapeutic substance contained in the container to an inner ear, and
the therapeutic substance container is configured to be located entirely within a middle ear cavity and/or the inner ear of a human.

15. The device of claim 14, wherein:
the device is configured to actively control itself to deliver therapeutic substances contained in the container to the inner ear.

16. The device of claim 14 or 15, wherein: The device of claim 14, wherein:
the device is configured to release the therapeutic substance contained in the container through active transportation.

17. The device of claim 14, wherein:
the device is configured to enable self-regulatory release of therapeutic substance contained in the container into the inner ear.

18. The device of claim 14, wherein:
the tissue interface is located in bone establishing a barrier between a middle ear and the inner ear;
the tissue interface has been implanted in the bone for at least 1 month; and
the device has no componentry configured to electrically and/or mechanically stimulate tissue to evoke a sensory response of the human.

19. The device of claim 14, wherein:
the device includes a fluid valve between fluid of the inner ear and an outside of the inner ear; and
the device is configured so that the valve can be actively adjusted to control an amount of therapeutic substance to be released into the inner ear from the container.

20. The device of claim 14, wherein:
the device is configured to be actively controlled remotely to deliver the therapeutic substance to an inner ear.

21-39. (canceled)
